(19) United States
(12) Patent Application Publication
(10) Pub. No.: US 2016/0279245 A1
(43) Pub. Date: Sep. 29, 2016

(54) TOPICAL FORMULATIONS, SYSTEMS, AND METHODS

(71) Applicant: NUVO RESEARCH INC., Mississauga (CA)

(72) Inventors: Wade Hull, Kaysville, UT (US); Tejas Desai, Mississauga (CA)

(21) Appl. No.: 15/033,467

(22) PCT Filed: Oct. 31, 2014
(86) PCT No.: PCT/CA2014/051049

§ 371 Date: Apr. 29, 2016

Related U.S. Application Data
(60) Provisional application No. 61/898,653, filed on Nov. 1, 2013.

(51) Int. Cl. A61K 47/20; A61K 31/167; A61K 31/245; A61K 31/196; A61F 7/03; A61K 47/32; A61K 47/26; A61K 47/14; A61K 9/70; A61K 9/00; A61K 31/573

(52) U.S. Cl. CPC A61K 47/20; A61K 9/0014; A61K 31/167; A61K 31/245; A61K 31/196; A61K 31/573; A61K 47/32; A61K 47/26; A61K 47/14; A61K 9/7023; A61F 7/03; A61F 2007/0226

(57) ABSTRACT

The present disclosure is drawn to topical formulations, transdermal systems, and related methods. In one embodiment, a topical formulation is provided that includes a drug such as a local anesthetic, an NSAID, or a corticosteriod; and sodium lauryl sulfoacetate. The topical formulations can have enhanced physical and/or chemical stability as compared to similar formulations without sodium lauryl sulfoacetate.

This application claims priority to or the benefit of U.S. Provisional Application Ser. No. 61/898,653, filed on Nov. 1, 2013, which is incorporated herein by reference.

### FIELD

Described are topical formulations, transdermal systems, and related methods. The topical formulations include sodium lauryl sulfoacetate for enhancing physical and/or chemical stability.

### BACKGROUND

Topical formulations for application to the skin can be useful in cosmetic applications or for treating conditions of the upper skin layers, and for transdermal administration of active agents to the local tissue underlying the skin or into the blood for systemic distribution. Use of a topical formulation containing a pharmaceutical agent is advantageous in that it avoids first-pass metabolism, circumvents gastrointestinal (“GI”) absorption, can allow delivery of an active ingredient with a relatively short biological half-life and/or a narrow therapeutic window, can facilitate uniform plasma dosing of the active ingredient, and/or can improve user compliance. In spite of these advantages, transdermal administration from transdermal patches is usually limited to small lipophilic drugs, e.g., scopolamine, fentanyl, estradiol, nitroglycerine, nicotine, and testosterone. Skin can impede the flux of exogenous molecules so as to provide a strong barrier to molecular delivery, particularly agents such as pharmaceutical agents, and thus, transdermal drug administration is difficult since skin is an excellent diffusion barrier. Over 300 substances have been identified as excipients for drug products but surprisingly few have been successfully developed into commercial formulations. U.S. Pat. Nos. 7,795,309, 8,343,962 and 8,513,304 disclose topical formulations requiring at least two excipients. Many excipients are irritating to the cells of the epidermis which can limit both the choice and concentration of the excipient suitable for topical formulations. Other excipients have a negative impact on the stability of the formulation, for example, changing the pH of the composition over time, degrading the active component of the formulation, or causing unusual changes in color or odor of the composition. These factors may impact both the product's shelf life and its administration regimen. Thus, there is a strong need to develop new topical compositions with improved chemical and/or physical stability.

### SUMMARY

With this background in mind, the present disclosure is drawn to topical formulations, transdermal systems, and related methods. In one embodiment, the application provides topical formulations and transdermal systems with improved chemical and/or physical characteristics. In another embodiment, the application provides topical formulations and transdermal systems with improved stability. In a further embodiment, the application provides topical formulations and transdermal systems with improved commercial shelf-life. In another embodiment, the application provides stabilized topical formulations and transdermal systems for long periods of topical administration.

In one embodiment, the application provides a topical formulation comprising a drug such as a local anesthetic, a non-steriodal anti-inflammatory drug (NSAID), or a corticosteriod; and sodium lauryl sulfoacetate (SLSA). In another embodiment, the application provides a topical formulation comprising a local anesthetic, SLSA, and one or more other excipients. In a further embodiment, the application provides a topical formulation comprising a drug, SLSA, water, and an excipient that is other than N-lauroyl sarcosine, sodium octyl sulfate, methyl laurate, isopropyl myristate, oleic acid, and glyceryl oleate. In a further embodiment, the topical formulation is free of N-lauroyl sarcosine, sodium octyl sulfate, methyl laurate, isopropyl myristate, oleic acid, and glyceryl oleate. In still another embodiment, the application provides a topical formulation comprising a drug and SLSA, wherein the presence of SLSA improves the physical and/or chemical stability of the formulation for at least about one, at least about three, at least about six, at least about 12, or about at least 24 months. In yet a further embodiment, the presence of SLSA provides improved chemical stability of the formulation. In another embodiment, the presence of SLSA provides improved physical stability of the formulation. In another embodiment, the application provides a stabilized topical formulation comprising a drug and SLSA, wherein the stabilized topical formulation has improved physical and/or chemical characteristics. In each of these formulations, water is also typically present.

In another embodiment of the present disclosure, a system for transdermal delivery of a drug such as a local anesthetic, an NSAID or a corticosteroid is provided that includes the topical formulation as described herein and a heating component capable of or adapted for heating the skin surface to a temperature of 32° C. to 47° C.

In another embodiment, a method of therapeutically delivering a drug can comprise applying a system for transdermal delivery of a drug to a skin surface of a subject. The system can comprise a topical formulation including the drug, sodium lauryl sulfoacetate, and water. The method can also include maintaining the system on the skin surface for a period of time to provide a therapeutic effect to the subject. For example, a local anesthetic, an NSAID, or a corticosteriod can be maintained on the skin surface for a sufficient period of time to achieve a therapeutic effect, e.g., local anesthetic for at least 15 minutes to numb the skin, local anesthetic or NSAID for at least one or at least two hours to achieve an analgesic effect, or a corticosteriod for a sufficient period of time to improve a skin condition. This method can also include applying a system for delivering the drug to the skin surface of a subject with a heating component capable of or adapted for heating the skin surface to a temperature of 32° C. to 47° C., as well as the topical formulation described herein, e.g., comprising a local anesthetic, an NSAID, or corticosteriod; SLSA; and water.

In still another embodiment, a method of improving the chemical and/or physical stability of topical formulations containing a drug such as a local anesthetic base and water is provided. The method includes admixing SLSA with the local anesthetic base and water to form the topical formulation. In another embodiment, a process for stabilizing a topical formulation comprising admixing a stabilizing amount of sodium lauryl sulfoacetate with a drug and water to form a stabilized topical formulation. In yet another embodiment the application provides for a stabilized topical formulation derived from the process provided herein.

### BRIEF DESCRIPTION OF THE DRAWINGS

Reference will now be made to the exemplary embodiments illustrated, and specific language will be used herein to describe the same. It will nevertheless be understood that no limitation of the scope of the invention is thereby intended.

FIG. 1 is a plot of the physical stability (phase separation) at 18° C.-25° C. of exemplary formulations F1-F5 as disclosed herein.

FIG. 2 is a plot of the physical stability (phase separation) at 40° C.±2° C. of exemplary formulations F1-F5 as disclosed herein.

FIG. 3 is a plot of the chemical stability (formation of 4-BABA) of exemplary formulations F1-F5 as disclosed herein when stored at 25° C.±2° C.

FIG. 4 is a plot of the chemical stability (formation of 4-BABA) of exemplary formulations F1-F5 as disclosed herein when stored at 40° C.±2° C.

FIG. 5 is a plot of the viscosity of exemplary formulations F1-F5 as disclosed herein when stored at 25° C.±2° C.

FIG. 6 is a plot of the viscosity of exemplary formulations F1-F5 as disclosed herein when stored at 40° C.±2° C.

FIG. 7 is a plot of the physical stability (phase separation) of exemplary formulations F6-F12 as disclosed herein when stored at 25° C.±2° C.

FIG. 8 is a plot of the physical stability (phase separation) of exemplary formulations F6-F12 when stored at 40° C.±2° C. as disclosed herein.

FIG. 9 is a plot of the chemical stability (formation of 4-BABA) of exemplary formulations F6-F12 as disclosed herein when stored at 25° C.±2° C.

FIG. 10 is a plot of the chemical stability (formation of 4-BABA) of exemplary formulations F6-F12 as disclosed herein when stored at 40° C.±2° C.

FIG. 11 is a plot of the viscosity of exemplary formulations F6-F8 and F10-F12 as disclosed herein when stored at 25° C.±2° C. (F9 was excluded from the graph because its values are much larger and distort the plot).

FIG. 12 is a plot of the viscosity of exemplary formulations F6-F8 and F11-F12 as disclosed herein when stored at 40° C.±2° C. (F9 and F10 were excluded from the graph because its values are much larger and distort the plot).

FIG. 13 is a plot of the chemical stability (formation of 4-BABA) of exemplary formulations F13-F15 as disclosed herein when stored at 2° C.-8° C.

FIG. 14 is a plot of the chemical stability (formation of 4-BABA) of exemplary formulations F13-F15 as disclosed herein when stored at 25° C.±2° C.

FIG. 15 is a schematic representation of an exemplary transdermal system of the present disclosure.

### DETAILED DESCRIPTION

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise. Thus, for example, reference to “a local anesthetic” includes reference to one or more of such local anesthetics.

The terms “long periods” or “longer periods” as used herein means at least about two hours, about three hours, about four hours, about five hours, about six hours, about seven hours, about eight hours, about nine hours, about ten hours, about 11 hours, about 12 hours, or more and any fractions in between.

The term “active agent” as used herein indicates a compound or mixture of compounds that, when added to a composition, tends to produce a particular therapeutic effect.

“Skin” is defined to include human skin (intact, diseased, ulcerous, or broken) as well as mucosal surfaces that are usually at least partially exposed to air such as lips, genital, anal, nasal, and oral.

The local anesthetic formulations and systems disclosed herein can be used both as anesthetics as well as analgesics. It is understood that “anesthesia,” refers to preventing pain before it happens, such as preventing a pain caused by needle stick. A formulation used for analgesic purposes or to provide “analgesia” refers to the formulations ability to reduce or eliminate an existing pain, e.g., musculoskeletal pain; muscle pain; back pain; nerve entrapment pain; neuroma pain; headache associated with neuralgia such as occipital neuralgia or trigeminal neuralgia; connective tissue pain such as iliotibial band pain, blood vessel pain, tendinopathy pain, medial tibial stress syndrome pain, bursitis, etc.; arthritis pain such as osteoarthritis pain or rheumatoid arthritis pain; pain associated with injury such as fracture, severance, break, sprain, strain, tear, point pain (e.g., trigger point pain or hit point pain), focal pain, or bruise; or combinations of these pains. The NSAID formulations and systems disclosed herein may also be used to reduce or eliminate an existing pain.

The terms “controlled heating” and “controlled heat” are defined as heat application that is capable of heating a skin surface or a drug formulation (and typically both) to pre-determined narrow temperature range for a predetermined duration. A controlled heating device that can be used in accordance with systems and methods of the present disclosure can be configured to generate heat (typically relatively promptly) when activated. Controlled heating can be achieved through special design of the heating component. For example, controlled heating can be achieved through the use of a properly configured heating element(s) including an exothermic chemical composition. Considerations in generating controlled heat with an exothermic heating component can include using proper ratios and exothermic chemical compositions, as well as physical constraints put on the exothermic chemical compositions, e.g., limiting air flow or oxygen contact, spatial configuration of individual heating elements, conductivity of materials used with the exothermic chemical composition, etc. In one embodiment, the heating component can provide heat at a temperature greater than body temperature, but less than a temperature that would cause irreversible skin damage, e.g., burn the skin. An exemplary temperature range that can be implemented for use can be from about 32° C. to about 47° C. In one embodiment, another temperature range can be from about 34° C. to about 44° C. or from about 35° C. to about 42° C. Other desired temperature ranges include from about 38° C. to about 42° C. or from about 36° C. to about 40° C.

The term “pharmaceutically acceptable” means compatible with the treatment of animals, in particular mammals, and most particularly, humans.

As used herein, the term “stabilized topical formulation” means a formulation having a stabilizing amount of SLSA that provides improved chemical and/or physical stability as compared to a comparative formulation devoid of sodium lauryl sulfoacetate and replaced with an equivalent wt % of water.

As used herein, the term “comparative formulation” is a formulation that is compositionally identical to a formulation prepared in accordance with the present invention, with the exception that the amount (wt %) of the sodium lauryl sulfoacetate is replaced with the same amount (wt %) of water.

As used herein, the term “free of” means that the composition comprises no detectable levels of N-lauroyl sarcosine, sodium octyl sulfate, methyl laurate, isopropyl myristate, oleic acid, and glyceryl oleate.

As used herein, the term “transdermal” means in the broadest sense through the skin.

As used herein the term “topical formulation” refers to a formulation that may be applied to skin or a mucosa surface. Topical formulations may, for example, be used to confer therapeutic benefit to a patient. Topical formulations can be used for both surface (non-transdermal) as well as transdermal (penetrating the skin or mucosal surface) administration of substances.

The term “topical administration” is used in its conventional sense to mean delivery of a substance, such as a therapeutically active agent, to the skin, mucosal surface, or a localized region of the body. Topical administration of a local anesthetic drug, for example, may often be advantageously applied during or prior to, for example, a painful medical or cosmetic procedure or to numb or otherwise treat the skin, or to reduce or eliminate an already existing pain.

The term “transdermal administration” is used to mean administration through the skin or mucosal surface. Transdermal administration is often applied where systemic delivery of an active is desired, or alternatively, it may also be useful for delivering an active to tissues underlying the skin with minimal systemic absorption (i.e. localized delivery).

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, delaying or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. In addition to being useful as methods of treatment, the methods described herein may be useful for the prevention or prophylaxis of disease.

The methods described herein comprise administering to a subject a therapeutically effective amount of an active agent. A treatment or prophylactic course of therapy may consist of a single administration, or alternatively comprises a series of applications. The length of the treatment period depends on a variety of factors, such as the severity of the condition, the age of the patient, the concentration of active ingredient or agent, the activity of the compositions described herein, and/or a combination thereof. It will also be appreciated that the effective dosage of the agent used for the treatment or prophylaxis may increase or decrease over the course of a particular treatment or prophylaxis regime. Changes in dosage may result and become apparent by standard diagnostic assays known in the art. In some instances, chronic administration may be required. In some embodiments, the compositions are administered to the subject in an amount and for a duration sufficient to treat the patient.

The term “subject” as used herein includes all members of the animal kingdom, including mammals, and suitably refers to humans.

As used herein, the term “skin contact region” refers to an area wherein the topical formulation contacts the skin. The skin contact region can have a size measured by area, and can vary from about 2 cm² to about 200 cm², typically.

The term “water” as used herein as an ingredient in the compositions of the application refers to pharmaceutically-acceptable water.

The term “w/v” as used herein means the number of grams of a substance in 100 mL of a composition.

The term “w/w” as used herein means the number of grams of a substance in 100 g of a composition.

The term “v/v” as used herein means the number of mL of a substance in 100 mL of a composition.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein. For example, in one embodiment, the degree of flexibility can be within about ±10% of the numerical value. In another embodiment, the degree of flexibility can be within about ±5% of the numerical value. In a further embodiment, the degree of flexibility can be within about ±2%, ±1%, or ±0.05%, of the numerical value.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

As used herein, a plurality of drugs such as local anesthetics, NSAIDS, or corticosteriods, compounds, and/or heating mechanisms may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 mm to 2.0 mm” should be interpreted to include not only the explicitly recited values of about 0.01 mm to about 2.0 mm, but also to include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise (4-BABA percentages, for example, are are not in weight).

The present disclosure is drawn to various formulations, systems, and methods in the area of transdermal delivery. In one example, formulations, systems, and related methods can be topical formulations for either surface and/or transdermal administration. In one embodiment, a topical formulation is provided that includes a drug and sodium lauryl sulfoacetate (SLSA). The drug may be selected from any drug known in the art including the active agents disclosed in U.S. Pat. No. 8,343,962 (column 5, line 3, to column 33, line 25), which is incorporated herein by reference. Without being bound by theory, drugs or compositions that are difficult to formulate or to stabilize may benefit from the presence of SLSA by improved physical and/or chemical stability. In one embodiment, a topical formulation is provided that includes a non-steroidal anti-inflammatory drug (NSAID) and sodium lauryl sulfoacetate (SLSA). In another embodiment, a topical formulation is provided that includes a corticosteroid and sodium lauryl sulfoacetate (SLSA). In a further embodiment, a topical formulation is provided that includes a local anesthetic and sodium lauryl sulfoacetate (SLSA).

Generally, any NSAID known in the art can be incorporated into topical formulations and systems disclosed herein. Non-limiting examples of such NSAIDs include acetaminophen, aspirin, bromefenac sodium, diclofenac, diclofenac potassium, diclofenac sodium, diflunisal, etodolac, fenoprofen calcium, flurbiprofen, ibuprofen, indomethacin, ketoprofen, magnesium salicylate, meclofenamate sodium, mefenamic acid, nabumetone, naproxen, naproxen sodium, oxaproxin, oxyohebutazone, phenylbutazone, piroxicam, rofecoxib, salsalate, sodium salicylate, sulindac, tometin sodium, valdexocib, and combinations thereof. In one embodiment, the NSAID can be a pharmaceutically acceptable salt of an NSAID. In a further embodiment, the local anesthetic can be a pharmaceutically acceptable base of an NSAID. In another embodiment, the NSAID can include diclofenac or ibuprofen or combinations thereof.

Generally, any corticosteroid known in the art can be incorporated into topical formulations and systems disclosed herein. Non-limiting examples of such corticosteroids include alclometasone dipropionate, amcinonide, beclomethasone dipropionate, betamethasone, betamethasone benzoate, betamethasone dipropionate, betamethasone valerate, budesonide, clobetasol propionate, desonide, desoximetasone, dexamethasone, diflorasone diacetate, fludrocortisone acetate, flunisolide, flurandrenolide, fluocinolone acetonide, fluocinonide, fluticasone propionate, halcinonide, halobetasol, hydrocortisone, hydrocortisone valerate, methylprednisolone, mometasone furoate, prednisolone, prednisone, triamcinolone, triamcinolone acetonide, and combinations thereof. In one embodiment, the corticosteroid can be a pharmaceutically acceptable salt of a corticosteroid. In a further embodiment, the corticosteroid can be a pharmaceutically acceptable base of a corticosteroid. In one embodiment, the corticosteroid can include clobetasol, halobetasol, betamethasone, triamcinolone acetonide and combinations thereof.

Generally, any local anesthetic known in the art can be incorporated into topical formulations and systems disclosed herein. Non-limiting examples of such local anesthetics include lidocaine, tetracaine, benzocaine, prilocaine, bupivacaine, dimethocaine, mepivacaine, procaine, ropivacaine, trimecaine, articaine, and combinations thereof. In one embodiment, the local anesthetic can be a pharmaceutically acceptable salt of a local anesthetic. In a further embodiment, the local anesthetic can be a pharmaceutically acceptable base of a local anesthetic. In another embodiment, the local anesthetic can include lidocaine, tetracaine, or combinations thereof. In another embodiment, the local anesthetic can include a eutectic mixture of lidocaine and tetracaine.

The drug such as a local anesthetic (in aggregate if more than one is used) can typically comprise from 10 wt % to about 50 wt % of the topical formulations disclosed herein, though concentrations outside of this range such as about 0.01 wt % to about 10 wt % can likewise be used. For example, the local anesthetic can be present at from 1 wt % to 50 wt % in some embodiments, or 2 wt % to 20 wt % in other embodiments. Alternatively, the local anesthetic can comprise about 20 wt % to about 45 wt % of the topical formulation. In another embodiment, the local anesthetic can comprise about 30 wt % to about 45 wt % topical formulation. In still another embodiment, the local anesthetic can comprise at least about 14 wt % of the topical formulation. In yet another embodiment, the local anesthetic can comprise at least about 30 wt % of the topical formulation. In a further embodiment the local anesthetic can comprise at least about 35 wt % of the topical formulation. In still a further embodiment, the local anesthetic can comprise about 40 wt % of the topical formulation. For clarity, with respect to these ranges, if a single local anesthetic is used, then that local anesthetic is used to determine the weight percentage. If multiple local anesthetics are present, the total concentration of all local anesthetics present is used to determine the weight percentage.

The sodium lauryl sulfoacetate (SLSA) can be present in the composition in amounts up to 50 wt %. More typically, the total amount of the SLSA is up to about 40 wt %. Even more often, the total concentration or percentage of the SLSA is in the range of from about 0.1 wt % to about 35 wt %. Even more typically, the total concentration or percentage of the SLSA is in the range of from about 0.1 wt % to about 30 wt %. In still other examples, the total concentration of the SLSA is in the range of from about 0.1 wt % to about 25 wt %, or from about 0.1 wt % to about 20 wt %. Further, the total concentration of the SLSA can be in the range of from about 0.1 wt % to about 29.5 wt %, about 0.5 wt % to about 29.5 wt %, about 1 wt % to about 29.5 wt %, about 0.1 wt % to about 19.5 wt %, about 0.5 wt % to about 19.5 wt %, 1 wt % to about 19.5 wt %, about 0.1 wt % to about 15 wt %, about 0.5 wt % to about 15 wt %, about 1 wt % to about 15 wt %, about 0.1 wt % to about 14.5 wt %, about 0.5 wt % to about 14.5 wt %, about 1 wt % to about 14.5 wt %, about 0.1 wt % to about 12.5 wt %, about 0.5 wt % to about 12.5 wt %, about 1 wt % to about 12.5 wt %, about 0.1 wt % to about 10 wt %, about 0.5 wt % to about 10 wt %, about 1 wt % to about 10 wt %, about 0.1 wt % to about 9 wt %, about 0.5 wt % to about 9 wt %, about 1 wt % to about 9 wt %, about 0.1 wt % to about 8 wt %, about 0.5 wt % to about 8 wt %, about 1 wt % to about 8 wt %, about 0.1 wt % to about 7 wt %, about 0.5 wt % to about 7 wt %, about 1 wt % to about 7 wt %, about 0.1 wt % to about 6 wt %, about 0.5 wt % to about 6 wt %, about 1 wt % to about 6 wt %, about 0.1 wt % to about 5 wt %, about 0.5 wt % to about 5 wt %, about 1 wt % to about 5 wt %, about 0.1 wt % to about 4 wt %, about 0.5 wt % to about 4 wt %, about 1 wt % to about 4 wt %, about 0.1 wt % to about 3 wt %, about 0.5 wt % to about 3 wt %, about 1 wt % to about 3 wt %, about 0.1 wt % to about 2 wt %, about 0.5 wt % to about 2 wt %, or from about 1 wt % to about 2 wt % per unit volume of the formulation. In another example, the total percentage of the SLSA is in the range of from about 0.1 wt % to about 7.5 wt %, 0.5 wt % to about 7.5 wt %, from about 0.1 wt % to about 5.5 wt %, from about 0.5 wt % to about 5.5 wt %, from about 0.1 wt % to about 3 wt %, or even from about 0.5 wt % to about 3 wt %. In other examples, the total concentration of the SLSA is in the range of from about 0.1 wt % to about 30 wt % or even 0.5 wt % to about 15 wt %. In one example, the weight ratio of amount of the drug such as a local anesthetic, NSAID, or corticosteriod (total) to the amount of the sodium lauryl sulfoacetate can be about 100:1 to 1:10. In another example, the weight ratio of the amount of the local anesthetic, NSAID, or corticosteriod (total) to the amount of the sodium lauryl sulfoacetate can be about 100:1 to about 10:1. In a further embodiment, the weight ratio of the amount of the local anesthetic, NSAID, or corticosteriod (total) to the amount of the sodium lauryl sulfoacetate can be from about 80:1 to about 20:1. In yet another example, the weight ratio of the amount of the local anesthetic, NSAID, or corticosteriod (total) to the amount of the sodium lauryl sulfoacetate can be from about 10:1 to about 1:10. In yet another example, the weight ratio of the amount of the local anesthetic, NSAID, or corticosteriod (total) to the amount of the sodium lauryl sulfoacetate can be from about 9:1 to about 1:9. In yet still another example, the weight ratio of the amount of the local anesthetic, NSAID, or corticosteriod (total) to the amount of the sodium lauryl sulfoacetate can be from about 10:1 to about 1:1.

The topical formulations of the present disclosure can include SLSA as well as one or more additional excipients. Generally, any pharmaceutically acceptable excipient can be used. In one aspect, the topical formulation can have one or more pharmaceutically acceptable excipient(s) other than N-lauroyl sarcosine, sodium octyl sulfate, methyl laurate, isopropyl myristate, oleic acid, glyceryl oleate, and combinations thereof. In another aspect, the topical formulation can be free of the excipients N-lauroyl sarcosine, sodium octyl sulfate, methyl laurate, isopropyl myristate, oleic acid, and glyceryl oleate.

The presence of SLSA in the formulations can enhance the physical and chemical stability of topical formulations containing local anesthetics, NSAIDs, or corticosteriods. In one embodiment, the application provides a stabilized topical formulation comprising a stabilizing amount of SLSA. Local anesthetics, particularly local anesthetic bases, are often oily and can separate from an aqueous medium when included in topical formulation such as those disclosed and stored for periods of as little as one to two weeks at room temperature. Other drugs or ingredients that are difficult to formulate may also result in compositions that lack stability. Long storage periods can result in increased phase separation in the formulations. One of the surprising benefits of the stabilized topical formulations disclosed herein is the improved physical stability of the formulations when stored. For example, in one embodiment, the stabilized topical formulation can have less phase separation after being stored for one week at 25° C. when compared to a comparative formulation devoid of SLSA (with an equivalent wt % of water replacing the SLSA). In a further embodiment, the stabilized topical formulation can have less phase separation after being stored for two weeks, three weeks, four weeks, 8 weeks or 12 weeks at 25° C. when compared to a comparative formulation devoid of SLSA (with an equivalent wt % of water replacing the SLSA). In another embodiment, the stabilized topical formulation can have less phase separation after one week when stored at about 40° C. when compared to a comparative formulation devoid of SLSA (again with an equivalent wt % of water replacing the SLSA). In a further embodiment, the stabilized topical formulation can have less phase separation after being stored for two weeks, three weeks, four weeks, 8 weeks or 12 weeks at 40° C. when compared to a comparative formulation devoid of SLSA (with an equivalent wt % of water replacing the SLSA). In still another embodiment, the stabilized topical formulation can have a phase separation that is at least 10% less, at least 20% less, at least 30% less, at least 50% less, at least 75% less, at least 90% less or, or even at least 100% less after two weeks when stored at about 40° C. when compared to a comparative formulation devoid SLSA (again as in each of these examples, replacing the removed SLSA with an equivalent wt % of water). In further embodiment, the stabilized topical formulation can have a phase separation that is at least 10% less, at least 20% less, at least 30% less, at least 50% less, at least 75% less, at least 90% less or, or even at least 100% less after two weeks when stored at about 25° C. when compared to a comparative formulation devoid SLSA (again as in each of these examples, replacing the removed SLSA with an equivalent wt % of water).

Alternatively, or in addition to the improvements in physical stability, the stabilized topical formulations of the present disclosure can provide surprising improvements in chemical stability of the local anesthetics contained therein, for example, when the local anesthetic includes a local anesthetic ester such as tetracaine. Local anesthetic esters or ester-type local anesthetics have been known to be susceptible to chemical degradation which can result in reduced concentrations of the local anesthetic and/or increase in certain impurities. For example, tetracaine is known to degrade via hydrolysis in the presence of water to form certain impurities (the primary hydrolysis product or impurity being isp-butyl amino benzoic acid (4-BABA)).

Tetracaine is a particularly difficult local anesthetic to stabilize long term, especially in aqueous formulations. The presence of the lidocaine in a eutectic mixture can provide some added stability, but it was surprising to discover that SLSA provided the added benefit of further stabilizing tetracaine, the local anesthetic ester, even when already admixed with lidocaine as part of a eutectic mixture. In one embodiment, the stabilized topical formulations disclosed herein can provide improved chemical stability for local anesthetic esters, such as tetracaine, such that, after being stored for a period of time, e.g., one, two, three, or four months, when stored at about 2° C.-8° C., at about 25° C., or at about 40° C., the formulation had a lower concentration of impurities associated with the degradation of the local anesthetic ester as compared to a comparative formulation devoid of SLSA (again with an equivalent amount of water added thereto to replace the SLSA).

In certain embodiments, by way of example, the topical formulations disclosed herein can provide improved stability for tetracaine such that, when the topical formulation includes tetracaine, after one, two, three, or four months stored at about 2° C.-8° C., at about 25° C. or at about 40° C., the stabilized formulation has a lower percentage of 4-BABA when compared to a comparative formulation devoid of SLSA (again with an equivalent wt % of water added thereto to replace the SLSA). The percentage of 4-BABA was determined by dividing the total amount of 4-BABA in the formulation by the total amount of the API (active pharmacuetical ingrediant) in the formulation and multiplying the resultant by 100. In another embodiment, when the stabilized topical formulation includes tetracaine, the formulation can have an amount of 4-BABA that is at least 5% lower after one, two, three, or four months stored at 25° C. compared to a comparative formulation where the SLSA is replaced with water and stored under the same conditions for the same amount of time. In a further embodiment, when the stabilized topical formulation includes tetracaine, after one, two, three, or four months stored at 25° C., the formulation has an amount of 4-BABA that is at least about 10%, at least about 20%, at least about 30%, at least about 40%, at least about 50%, or at least about 60% lower when compared to a comparative formulation devoid of SLSA. In yet another embodiment, when the stabilized topical formulation includes tetracaine, after one, two, three, or four months stored at 40° C., the formulation with SLSA has an amount of 4-BABA that is at least about 1%, at least about 2%, at least about 5%, at least about 15%, at least about 30%, or at least about 45% lower when compared to a comparative formulation devoid of SLSA. In other embodiments, the SLSA can be replaced with oleic acid or another similar ingredient other than water. This can be done to ensure it is the SLSA that is providing the additional stability, as tetracaine is sensitive to hydrolysis. In one embodiment where oleic acid is used to replace the SLSA, for example, the formulation with SLSA can still have an amount of 4-BABA that is at least 14% lower after one, two, or three months when stored at 25° C. when compared to a formulation where the concentration of the oleic acid is replaced with water. In yet another embodiment, the formulation with SLSA can have an amount of 4-BABA that is at least 2% lower after one, two, or three months stored at 40° C. when compared to a formulation where the concentration of the oleic acid is replaced with water.

In still other embodiments, by way of example, a portion of the weight percentage of the SLSA can be replaced with calcium phosphate dibasic anhydrous. Again, this can be done to ensure it is the SLSA that is providing the additional stability, as tetracaine is sensitive to hydrolysis. In these formulations, the embodiments having SLSA and a lesser weight percentage of water with a higher weight percentage of calcium phosphate exhibited at least about a 33% lower amount of 4-BABA after two months and at least about a 34% lower amount of 4-BABA after three months when stored at 5° C. and compared to the formulation without SLSA and a greater weight percentage of water. Replacing a portion of the water with calcium phosphate dibasic anhydrous in the formulation had little to no effect on the concentration of 4-BABA at less than two months when stored at 5° C. In formulations containing 3 wt % SLSA with and without the altered water and calcium phosphate weight percentages and formulations containing 1 wt % SLSA without the altered water and calcium phosphate weight percentages, the concentration of 4-BABA can often be at least about 20% lower initially and at least about 27% lower after one month when stored at about 5° C. In formulations having SLSA and a lesser weight percentage of water with a higher weight percentage of calcium phosphate, at least about a 31% lower amount of 4-BABA after one month, at least about a 27% lower amount of 4-BABA after two months, and at least about a 30% lower amount of 4-BABA after three months when stored at 25° C. can be achieved in ceratin examples. In formulations containing 3 wt % SLSA without the altered water and calcium phosphate weight percentages, the amount of 4-BABA can be often be at least about 20% lower initially, at least about 21% lower after one month, at least about 24% lower after two months, and at least about 17% lower after three months when stored at about 25° C. Based on this, it is noted that the weight percentage of water in the total formulation has some effect on the production of 4-BABA, but it still remains clear that the SLSA still has a positive impact on the chemical stability of tetracaine in the formlulations of the present disclosure. Similar percent reductions described above with respect to 4-BABA can also be achieved for other impurities associated with the degradation of local anesthetic esters, i.e. at least about 10%, at least about 20%, or at least about 30% reductions compared to comparative formulations devoid of SLSA.

With the above in mind, the present disclosure is drawn to topical formulations, transdermal systems, and related methods. In one embodiment, the application provides a topical formulation comprising a drug such as a local anesthetic, NSAID, or corticosteriod and sodium lauryl sulfoacetate (SLSA). In another embodiment, the application provides a topical formulation comprising a local anesthetic; SLSA; and one or more other excipients. In a further embodiment, the application provides a topical formulation comprising a a drug such as a local anesthetic, NSAID, or corticosteriod; SLSA; and an excipient that is other than N-lauroyl sarcosine, sodium octyl sulfate, methyl laurate, isopropyl myristate, oleic acid, and glyceryl oleate. In a further embodiment, the topical formulation is free of N-lauroyl sarcosine, sodium octyl sulfate, methyl laurate, isopropyl myristate, oleic acid, and glyceryl oleate. In still another embodiment, the application provides a topical formulation comprising a local anesthetic, NSAID, or corticosteriod; and SLSA, wherein the presence of SLSA improves the physical and/or chemical stability of the formulation for at least about one, at least about three, at least about six, at least about 12, or about at least 24 months. In yet a further embodiment, the presence of SLSA provides improved chemical stability of the local anesthetic.

Without being limited by theory, it is believed that during longer periods of dermal application the formulation may be exposed to certain elements that could negatively impact stability, e.g. moisture, sweat, additional periods of heating. In one embodiment, the presence of SLSA provides improved chemical and/or physical stability useful in treatments with longer periods of dermal administration. In another embodiment, the presence of SLSA may lead to the inclusion of increased concentrations of drug(s) or other components in the formulation. This is based on the theory that tolerances levels for impurities would be exceeded at the increased concentrations without the inclusion of SLSA. Alternatively, where a formulation has known impurities, even within the permitted tolerance level set by regulatory bodies, the inclusion of SLSA can improve the impurity profile of a formulation. Further, a combination of SLSA and a reduced concentration of drug(s) or other components in the formulation may lead to substantial reduction or even elimination of certain impurities that would have normally been present in the formulation in the absence of SLSA.

By improving the physical stability of topical formulations, manufacturing of compositions that comprise the topical formulation can be simplified. For instance, where the topical formulation is stored prior to incorporation into a transdermal system, e.g. heated patch, peel-forming formulation, or plaster, less or even no mixing (with respect to duration and frequency) of the formulation may be needed once the formulation has been prepared. Improved physical and chemical stability of topical formulations may also lead to increased commercial shelf life of the system. In one embodiment, the presence of sodium lauryl sulfoacetate (SLSA) can provide an improved commercial shelf-life. In another embodiment, the topical formulation of the invention can have an increased shelf life of at least about one month, at least about two months, at least about three months, at least about six months, at least about nine months, or at least about 12 months compared to a comparative formulation devoid of sodium lauryl sulfoacetate stored under the same conditions. In another embodiment, the shelf life of the topical formulation can be at least about 27 months, at least about 30 months, at least about 33 months, or at least about 36 months.

The topical formulations can also include other components in addition to the drug and SLSA. Examples of additional compounds that can be included in the topical formulations include water, thickening, gelling and/or solidifying polymers, fatty acid esters, parabens, solvents, carriers and the like. In one embodiment, the topical formulation can include water, and in some case, the water can be the ingredient that is present in the single greatest concentration. Generally, the water can be present in amounts of about 25 wt % to about 80 wt %. In one embodiment, the water can be present in the formulation in an amount of about 35 wt % to about 70 wt %. In another embodiment, the water can be present in an amount of about 40 wt % to about 75 wt %. In yet another embodiment, the water can be present in an amount of about 35 wt % to about 50 wt %. In still another embodiment, the water can be present in an amount of about 40 wt % to about 50 wt %.

Polymers can also be included, including, without limitation, polyvinyl alcohol, (PVA), Gantrez ES-425 (a monobutyl ester of the copolymer of methyl vinyl ether and maleic anhydride in ethanol), poly(2-hydroxyethyl methacrylate), Plastoid B (a neutral copolymer based on butyl methacrylate and methyl methacrylate), and/or Eudragit S100 (anionic copolymer based on methacrylic acid and methyl methacrylate). In one specific embodiment, the topical formulation can include a polyvinyl alcohol. Generally, the polymer can comprise about 0.1 wt % to about 15 wt % of the formulation, from about 5 wt % to about 15 wt % of the formulation, or from about 6 wt % to about 12 wt % of the formulation.

Non-limiting examples of fatty acid esters that can be present include sorbitan monopalmitate, sorbitan monolaurate, sorbitan monomyristate, sorbitan monooleate, sorbitan monolinoleate, and combinations thereof. In one embodiment, the formulation can include sorbitan monopalmitate. The fatty acid ester can comprise about 0.1 wt % to about 10 wt % of the formulation, from about 1 wt % to about 5 wt % of the formulation, or from about 3 wt % to about 4 wt % of the formulation.

Parabens that may be included in the topical formulation include methylparaben, propylparaben, ethylparaben, butylparaben, isobutylparaben, isopropyl paraben, and/or benzyl paraben. In one embodiment, the topical formulation can include methylparaben, propyl paraben, or combinations thereof. The paraben can comprise about 0.01 wt % to about 0.6 wt % of the topical formulation. In one embodiment, the paraben can comprise about 0.01 wt % to about 0.5 wt % of the topical formulation.

Other suitable carriers that may be used in the topical formulations discussed herein are known in the art and include, but are not limited to, solubilizers such as C₂ to C₈ straight or branched chain alcohols, diols and triols, moisturizers and humectants such as glycerine, amino acids and amino acid derivatives, polyaminoacids and derivatives, pyrrolidone carboxylic acids and their salts and derivatives, surfactants such as sodium laureth sulfate, sorbitan monolaurate, emulsifiers such as cetyl alcohol, stearyl alcohol, thickeners such as methyl cellulose, ethyl cellulose, hydroxymethylcellulose, hydroxypropylcellulose, polyvinylpyrrolidone, polyvinyl alcohol and acrylic polymers. The topical formulation may also include propylene glycol. The propylene glycol may be present in the formulation at from about 1 wt % to about 25 wt %. Additionally the topical formulation may also include ethanol and/or polyethylene glycol 300, or other similar low molecular weight alcohol. The ethanol may be present in the formulation at from about 1 wt % to about 25 wt %. The polyethylene glycol 300 or other low molecular weight alcohol may be present in the range at from about 1 wt % to about 80 wt %. In addition, the topical formulation may include at least one moisturizer/humectant. Other examples of suitable excipients, such as binders and fillers, are listed in Remington's Pharmaceutical Sciences, 18th Edition, Ed. Alfonso Gennaro, Mack Publishing Co. Easton, Pa., 1995 and Handbook of Pharmaceutical Excipients, 3rd Edition, Ed. Arthur H. Kibbe, American Pharmaceutical Association, Washington D.C. 2000.

The topical formulations of the present disclosure may also include one or more skin care active agents. “Skin care active agents” means all compounds or substances now known or later demonstrated to provide benefit when applied to skin and all compounds now claimed or in the future claimed to provide benefit when applied to skin. Skin care active agents may provide benefits, or claimed benefits, in areas such as one or more of wrinkle removal or wrinkle reduction, firming of skin, exfoliation of skin, skin lightening, treatment of dandruff, treatment of acne, skin conditioning, development of tans and artificial tans, improvement of skin moisture content, improvement of skin barrier properties, control of sweat, anti-aging, reduction or avoidance of irritation and reduction or avoidance of inflammation. Examples of skin care active agents include molecules such as peptides, proteins, oligonucleotides, fullerenes as well as small molecules. Skin care active agents may be protease and/or enzyme inhibitors, anti-coenzymes, chelating agents, antibodies, antimicrobials, humectants, vitamins, skin protectants, antioxidants and/or skin soothing agents, plant extracts and the like. Examples of skin care active agents include but are not limited to vitamin C, vitamin E (alpha tocopherol), retinoids, soy derivatives (e.g. isoflavones), green tea polyphenols, alpha hydroxy acids (e.g. glycolic and lactic acids), beta hydroxy acids (e.g. salicylic acid), poly hydroxy acids, alpha lipoic acid, hemp oil (glycerides), niacinamide, dimethyl amino ethanol, coenzyme Q10, kinetin (plant growth hormone), dimethyl sulfone, and botulinum toxin. Other examples of skin care active agents may be found in, The Perricone Prescription by Nicholas Perricone, Harper Collins Publishers Inc., New York, 2002.

The topical formulations of the present disclosure can be utilized in the manufacture of systems for transdermal delivery of drugs such as local anesthetics, NSAIDs, or corticosteriods. In one aspect, a system for transdermal delivery of a local anesthetic, an NSAID, or a corticosteriod is provided that includes the topical formulation as disclosed herein, and a heating component capable of heating the skin surface to a temperature of 33° C. to 47° C., but more typically from 34° C. to 44° C., and often from 35° C. to 42° C. In one embodiment, the local anesthetic, NSAID, or corticosteriod can be present in a topical formulation applied to a skin contact portion of a transdermal patch. The systems can have any general shape or configuration known in the art including, but not limited to, substantially oval, round, square, triangular, or rectangular in shape, to name a few. The system can be such that the topical formulation has a skin contact region (i.e. an area that is configured to contact the skin surface of a subject) having an area of about 2 cm² to about 200 cm². In one embodiment, the topical formulation in the system can have a skin contact region having an area of about 7 cm² to about 150 cm². In another embodiment, the topical formulation in the system can have a skin contact region having an area of about 2 cm² to about 12 cm². In a further embodiment, the topical formulation in the system can have a skin contact region having an area of about 8 cm² to about 15 cm². In another embodiment, the topical formulation in the system can have a skin contact region having an area of about 15 cm² to about 25 cm². In yet another embodiment, the topical formulation in the system can have a skin contact region having an area of about 25 cm² to about 35 cm². While the skin contact region can have the areas described above, the system as a whole can have an area that contacts the skin that is greater than the skin contact region. In one embodiment, the area that contacts the skin for the system can be from 2 cm² to about 250 cm². In another embodiment, the area of the system that contacts the skin can be about 10 cm² to about 150 cm². In another embodiment, the area of the system that contacts the skin can be about 30 cm² to about 100 cm².

As described, the heating component of the systems can be configured to generate heat to a temperature of about 35° C. to about 47° C., about 36° C. to about 45° C., or about 36° C. to about 42° C. In a particular embodiment, the heating component can be configured to generate a controlled level of heat within any of these temperature ranges. The generation of the heat by the heating component can be by any means known in the art. In one embodiment, the heating component can include an exothermic chemical composition.

In one example, the heating component can generate heat by an exothermic oxidative chemical reaction. The chemical-based exothermic oxidation reaction can generate heat through the contact of the oxidative material, e.g. iron, with ambient air. U.S. Pat. No. 6,756,053, which is incorporated herein by reference in its entirety, describes examples of exothermic heating components and devices. The amount of exothermic chemical composition in the heating component can vary depending on the desired duration of heating and the size of the heating component. It can be beneficial to limit the amount of the exothermic chemical composition in the heating component, as a large amount of exothermic chemical composition can cause the heating component to be excessively large or cumbersome and impractical for use. In one embodiment, the heating device can include no more than 2 grams of an exothermic chemical composition and can be configured to heat an area of skin greater than about 8 cm². In another embodiment, the heating device can include 1.3 grams of an exothermic chemical composition and can be configured to heat an area of skin greater than about 10 cm². In a further embodiment, the heating device can include 2.6 grams of an exothermic chemical composition and can be configured to heat an area of skin greater than about 20 cm². In still a further embodiment, the heating device can include 5.2 grams of an exothermic chemical composition and can be configured to heat an area of skin greater than about 40 cm². In yet another embodiment, the heating device can include no more than 4.5 grams of an exothermic chemical composition and can be configured to heat an area of skin greater than about 25 cm².

In addition to the oxidizable component, the exothermic heating composition can further include activated carbon, salt (such as sodium chloride), and water. In one aspect, a water-retaining substance, such as vermiculite or wood powder, can also be included in the heating component. Depending on the configuration of the heating device, when stored for extended period of time the exothermic heating components can generate gas (believed to be methane and hydrogen) which can cause the packaging in which the exothermic heating component is present to puff up, which in turn can cause complications and problems with respect to storage and transportation. Furthermore, the inclusion of certain amounts of sulfur-containing compounds, such as elemental sulfur, sulfates, sulfites, sulfides, or thiosulfates, salts, etc., can reduce or eliminate this gas generation problem when included in the packaging.

Water content in the exothermic chemical composition can have an impact on the heating temperature profile of the heating device. The weight ratio of water to the rest of the ingredients in the exothermic heating component can be in the range of about 1:2.6 to about 1:5.0, though this range is not intended to be limiting. In one aspect, the exothermic chemical composition of the heating component can be manufactured in a manner so as to only have access to ambient oxygen through the holes in a cover that can be made of air-impermeable material. In this way, the flow rate of oxygen from ambient air into the exothermic chemical composition, which in turn can be a factor that can affect the amount and rate of heat generated by the heating component and the temperature of the skin surface on which the analgesic system is applied. Other factors which can influence the temperature and heat generation of the heating component can be the size of the heating component, the amount of the exothermic chemical composition in the heating component, the number and configuration of holes in the heating component's air impermeable cover material, etc.

The exothermic chemical composition can be formulated into a layer having an exothermic material disposed therein. In one embodiment, the system can include an air impermeable layer disposed on an upper surface of the chemical composition layer and can have one or more holes therein. In another embodiment, the system can include an activation tab removably adhered to an upper surface of the air impermeable layer and being configured to cover the one or more holes in the air impermeable layer and inhibit the passage of air through the holes prior to removal of the activation tab. In still another embodiment, the system can include an adhesive layer disposed on a lower surface of one or both of the exothermic chemical composition layer and the lower surface of the air impermeable layer, said adhesive layer being configured to adhere the system to a skin surface.

In addition to being formulated to be included in transdermal systems such as those described above, the topical formulations of the present invention may be formulated by those skilled in the art as liquids, solutions, emulsions, creams, lotions, suspensions, triturates, gels, plasters, peels, jellies, foams, pastes, ointments, shampoos, adhesives, other more traditional patches without a heating component, or the like.

The local anesthetic topical formulations and systems disclosed herein can be utilized for the analgesic treatment of pain in a subject, or alternatively, for the anesthesia treatment of the skin prior to a painful medical procedure, e.g., needle stick, incision, skin treatment, or the like. The NSAID formulations and systems disclosed herein may also be used for the analgesic treatment of pain. The corticosteriod formulations and systems disclosed herein may be used to treat dermal conditions including but not limited to include, pruritus, psoriasis, dermatitis, herpetiformis, and eczema.

Thus, in one embodiment, a method of treating or preventing pain can include applying a topical formulation or system for delivering a local anesthetic or NSAID to a skin surface of a subject experiencing pain. Particularly in the case of numbing the skin prior to a painful medical procedure with a local anesthetic, the method can further include maintaining the topical formulation or system can be maintained on the skin surface of the subject for a period of time of at least 15 minutes, at least 20 minutes, at least 30 minutes, at least one hour, or at least two hours, such that the topical formulation is in contact with the skin surface and the heating component is activated to apply the temperature to the topical formulation and/or the skin surface. On the other hand, methods preventing pain analgesically can be carried out using local anesthetics and/or NSAIDS. The heating component can begin heating at about the same time as the system is applied to the skin surface. In another embodiment, formulation and/or heat can be maintained on the skin surface for a period of time of at least about one hour. In still another embodiment, the system or formulation (with or without heat) can be maintained on the skin surface for a period of time of at least about two hours, four hours, six hours, eight hours, ten hours, 12 hours, 24 hours, etc. In one embodiment, the system can be applied for a period of time and then removed, and then optionally, reapplied with a new patch (with or without heat), plaster, or peel. In the case of analgesia, pain relief can continue for a period of hours, in some cases days, following the removal of the system from the skin surface. In still another example, if the drug is a corticosteriod, the system can be applied, with or without heat in the form of a patch, plaster, or peel, to treat skin disorders, such as pruritus, psoriasis, dermatitis, herpetiformis, or eczema. Application times and cycles can vary, but usually application times of at least about one hour, two hours, four hours, eight hours, 12 hours, or 24 hours can be effective.

In one embodiment, the treatment is administered once a day. In another embodiment, the treatment is administered twice a day. In still another embodiment, the treatment is administered three times a day. In yet another embodiment, the treatment is administered four times a day. In a further embodiment, the treatment is administered one to two times a day for one, two, three, four, five, six, or seven days. In still a further embodiment, the treatment is administered at least once a day for a longer term such as one, two, three, four, five, six, seven, eight, nine, ten, 11, or 12 days, etc. In an even further embodiment, the treatment is administered at least once a day until the condition has ameliorated to where further treatment is not necessary. In another embodiment, the persistence of pain is reduced for a period of time following administration of the topical formulation, for example, days, weeks or months. Thus, pain relief can remain after the topical formulation or patch is removed in many instances.

In another embodiment, the treatment is administered at least once per week. In another embodiment, the treatment is administered twice per week. In still another embodiment, the treatment is administered three times per week. In yet another embodiment, the treatment is administered four times per week. In yet another embodiment, the treatment is administered five times per week. In yet another embodiment, the treatment is administered six times per week. In a further embodiment, the treatment is administered one to six times per week for one, two, three, four, five, six, or seven weeks. In still a further embodiment, the treatment is administered at least once per week for a longer term such as one, two three, four, five, six, seven, eight, nine, ten, 11, or 12 weeks, etc. In an even further embodiment, the treatment is administered at least once per week until the condition has ameliorated to where further treatment is not necessary. Thus, pain relief can remain after the topical formulation or patch is removed in many instances.

When not presented and applied to a skin surface in the form of a transdermal delivery system (e.g. patch), the present topical formulation may be applied to the skin by any method known in the art including, but not limited to: an aerosol, spray, pump-pack, brush, swab, or other applicator. The applicator provides either a fixed or variable metered dose application such as a metered dose aerosol, a stored-energy metered dose pump or a manual metered dose pump. In this example, the topical formulation can be applied to the skin of the human or animal covering a delivery surface area from about 5 cm² to about 800 cm², more typically from about 7 cm² to about 400 cm², and most typically from about 7 cm² to about 200 cm². The application can be performed by means of a topical metered dose spray combined with an actuator nozzle shroud which together accurately control the amount and/or uniformity of the dose applied. One function of the shroud is to keep the nozzle at a pre-determined height above, and perpendicular to, the skin to which the drug delivery system is being applied. This function may also be achieved by means of a spacer-bar or the like. Another function of the shroud is to enclose the area above the skin in order to prevent or limit bounce-back and/or loss of the drug delivery system to the surrounding environment. The drug delivery system may be a unit volume dispenser with or without a roll-on or other type of applicator. It may also be desirable to apply a number of dosages on untreated skin to obtain the desired result.

In yet another embodiment the topical formulation can be applied to the skin by any method using a peel-forming formulation (or peel). For example, these formulations can be used with the peels disclosed in U.S. Patent Publication Nos. US-2012-0,022,158, US-2011-0,015,229, and US 2005-0,276,842; and U.S. Pat. No. 6,528,086, each of which is incorporated herein by reference.

In some aspects, it can be useful or desirable to include additional pharmaceutically active agents in the disclosed compositions. Formulations comprising multiple active agents are contemplated. Such active agents are known in the art and discussed supra with reference to U.S. Pat. No. 8,343,962 (column 5, line 3, to column 33, line 25), which is incorporated herein by reference.

Embodiments of the invention will be described with reference to the following Examples which are provided for illustration purposes only and should not be used to limit the scope of or construe the invention.

### EXAMPLES

### Example 1

### Transdermal Delivery Formulations—Set 1

Several formulations were prepared according to embodiments of the present disclosure utilizing the compositional components set forth in Table 1. Each of the formulations was prepared in a batch at a batch size of 2 kg. All raw materials were stored at ambient conditions prior to manufacturing of the formulations. Generally, all formulations were manufactured as described below.

Specifically, an oil phase was prepared by heating and mixing the mixture of the active agents (local anesthetics) at about 50° C.±5° C. The parabens, polymer, and excipients were added sequentially in water while stirring in combination with homogenization at high shear force under heated conditions (−75° C.±5° C.). The temperature of the mixing vessel was then lowered and the oil phase mixed into water and homogenized at room temperature (25° C.±5° C.) until a product without any lumps or crystals was obtained. The final product was then tested for the physical-chemical properties and placed on stability at appropriate storage conditions. A control formulation was also prepared that did not include SLSA. The SLSA content in the control formulation was replaced with water. However, it is noted that in these specific examples, all of the formulations (F1-F5) include multiple excipients, and thus, for consistency across all examples, all excipients were removed and replaced with water to provide a common control.

TABLE 1

| | C1 | | | | | |
|---|---|---|---|---|---|---|
| | (Control) | F1 | F2 | F3 | F4 | F5 |
| Ingrediant | Wt % | | | | | |
| Lidocaine | 20 | 20 | 20 | 20 | 20 | 20 |
| Tetracaine | 20 | 20 | 20 | 20 | 20 | 20 |
| Polyvinyl Alcohol | 7.2 | 7.2 | 7.2 | 7.2 | 7.2 | 7.2 |
| Sorbitan Monopalmitate | 3 | 3 | 3 | 3 | 3 | 3 |
| Purified Water | 49.68 | 47.68 | 47.68 | 47.68 | 43.68 | 41.68 |
| Methylparaben | 0.1 | 0.1 | 0.1 | 0.1 | 0.1 | 0.1 |
| Propylparaben | 0.02 | 0.02 | 0.02 | 0.02 | 0.02 | 0.02 |
| Isopropyl myristate | — | — | — | 1 | — | 5 |
| Oleic Acid | — | — | 1 | — | — | — |
| Glyceryl Oleate | — | 1 | — | — | 3 | — |
| Sodium Lauryl | — | 1 | 1 | 1 | 3 | 3 |
| Sulfoacetate | | | | | | |

### Example 2

### Physical Stability of the Formulations of Example 1

Each of the formulations and the control described in Example 1 were stored at ambient conditions (18° C. to 25° C. and ≦60% RH) and accelerated conditions (40° C.±2° C. and 75%±5% RH). The physical stability (i.e. the phase separation of the formulations) of samples stored at both ambient and accelerated conditions were tested at one, two, three, four, eight, 12, and 24 weeks.

The phase separation was measured by placing each of the formulations in a 125 ml graduated glass cylinder. The cylinders were stopped and placed at either 18° C.-25° C. or 40° C. Phase separation in the formulations were then determined by measuring the quantity of oil phase separated at the top of the cylinder in terms of milliliters.

The phase separation for each of the formulations are provided in Table 2A for the ambient temperature samples and Table 2B for the accelerated temperature samples.

TABLE 2A
Physical Stability at 18° C.-25° C.

| | | | | | | |
|---|---|---|---|---|---|---|
| Storage | C1 | F1 | F2 | F3 | F4 | F5 |

| | |
|---|---|
| (Weeks) | Phase Separation in (ml) |

| | | | | | | |
|---|---|---|---|---|---|---|
| 1 | 2 | 0 | 0 | 0 | 0 | 0 |
| 2 | 1 | 0 | 0 | 0 | 0 | 0 |
| 3 | 1 | 0 | 0 | 0 | 0 | 0 |
| 4 | 1 | 0 | 0 | 0 | 0 | 0 |
| 8 | 1 | 0 | 0 | 0 | 0 | 0 |
| 12 | 1 | 0 | 0 | 0 | 0 | 0 |
| 24 | 3.5 | 0 | 0 | 0 | 0 | 0 |

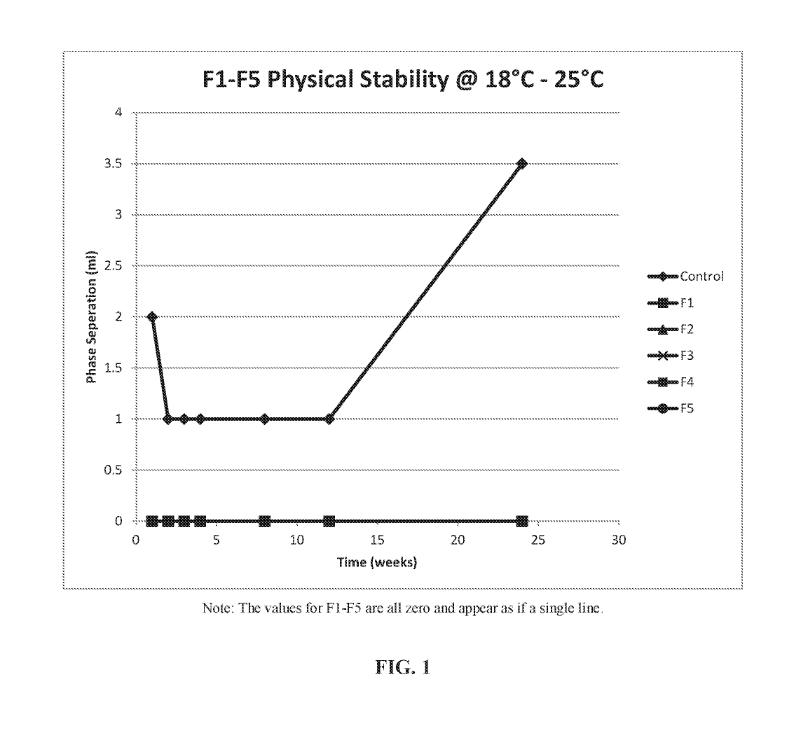

The physical stability at 18° C.-25° C. for formulations F1-F5 are plotted and shown in FIG. 1. Note that since all reported data points for F1-F5 are 0, a single line appears on the graph for the formulations of the present disclosure.

TABLE 2B
Physical Stability at 40° C. ± 2° C.

| | | | | | | |
|---|---|---|---|---|---|---|
| Storage | C1 | F1 | F2 | F3 | F4 | F5 |

| | |
|---|---|
| (Weeks) | Phase Separation (ml) |

| | | | | | | |
|---|---|---|---|---|---|---|
| 1 | 5 | 5 | 0 | 0 | 2 | 1 |
| 2 | 8 | 5 | 0 | 0 | 2 | 2 |
| 3 | 7 | 7 | 0 | 0 | 2 | 2 |
| 4 | 7 | 7 | 0 | 0 | 2 | 5 |
| 8 | 10 | 5 | 0 | 0 | 2 | 5 |
| 12 | 10 | 7 | 0 | 0 | 5 | 5 |
| 24 | 10 | 1 | 0 | 0 | 2 | 5 |

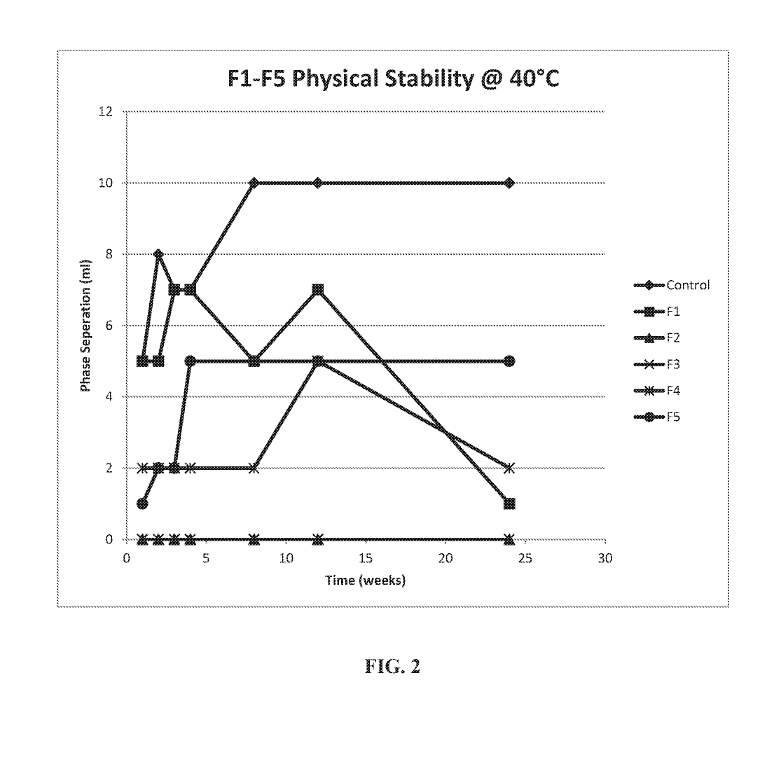

The physical stability at 40° C.±2° C. for formulations F1-F5 are plotted and shown in FIG. 2.

### Example 3

### Chemical Stability of the Formulations of Example 1

Each of the formulations and the control described in Example 1 were tested for the chemical stability of tetracaine following storage at long term conditions (25° C.±2° C./60%±5% RH) and accelerated conditions (40° C.±2° C. and 75%±5% RH). The chemical stability was measured in terms of the generation of the impurity 4-butylaminobenzoic acid (4-BABA), the primary hydrolysis degradation product of tetracaine. Specifically, tetracaine is known to break down into 4-BABA, so a lower concentration of this compound after a period of weeks demonstrates greater tetracaine stability over time. Samples in both long term and accelerated conditions were tested at three, eight, 12, and 24 weeks.

The chemical stability of the formulations was assessed by measuring the concentration of the active agents (lidocaine and tetracaine) and formation of degradation products, i.e. impurities, such as 4-BABA using High Performance Liquid Chromatographic (HPLC) method. The HPLC method involved chromatographic separation by mobile phase gradient and C 18 analytical column and quantification of each component by ultraviolet (UV) detector.

Results for the chemical stability for each of the formulations are provided in Table 3A for the long term temperature samples and in Table 3B for the accelerated temperature samples.

Each of these tables also includes the value for the amount of water reduced in each of the formulations due to the inclusion of the SLSA with or without other excipients (e.g., isopropyl myristate, oleic acid, and/or glyceryl oleate), as well as calculations for the percent difference (%) in the amount of 4-BABA production of the particular formulation as compared to the 4-BABA production of the control over a test period.

TABLE 3A
Chemical Stability at 25° C. ± 2° C.

| Storage | C1 | F1 | F2 | F3 | F4 | F5 |
|---|---|---|---|---|---|---|
| (Weeks) | 4-BABA % | | | | | |
| 0 | 0.06 | 0.03 | 0.02 | 0.02 | 0.01 | 0.02 |
| 3 | 0.23 | 0.17 | 0.16 | 0.16 | 0.13 | 0.12 |
| 8 | 0.48 | 0.4 | 0.39 | 0.40 | 0.33 | 0.29 |
| 12 | 0.69 | 0.6 | 0.56 | 0.57 | 0.49 | 0.43 |
| 24 | 1.39 | 1.24 | 1.14 | 1.22 | 0.99 | 0.88 |
| About 52 | 2.78 | 2.27 | 2.26 | 2.26 | 1.93 | 1.62 |
| weeks* | | | | | | |
| Water | 0 | 2 | 2 | 2 | 6 | 8 |
| Reduction | | | | | | |
| % Change | 0 | −11% | −18% | −12% | −29% | −37% |
| in 4-BABA | | | | | | |
| at 24 weeks | | | | | | |

*data is extrapolated
Note:
4-BABA % is calculate as follows: 4-BABA level (w/w) divided by API (active pharmaceutical ingrediant) level (w/w) × 100. The w/w units cancel out and the unit of measurement is a percentage.

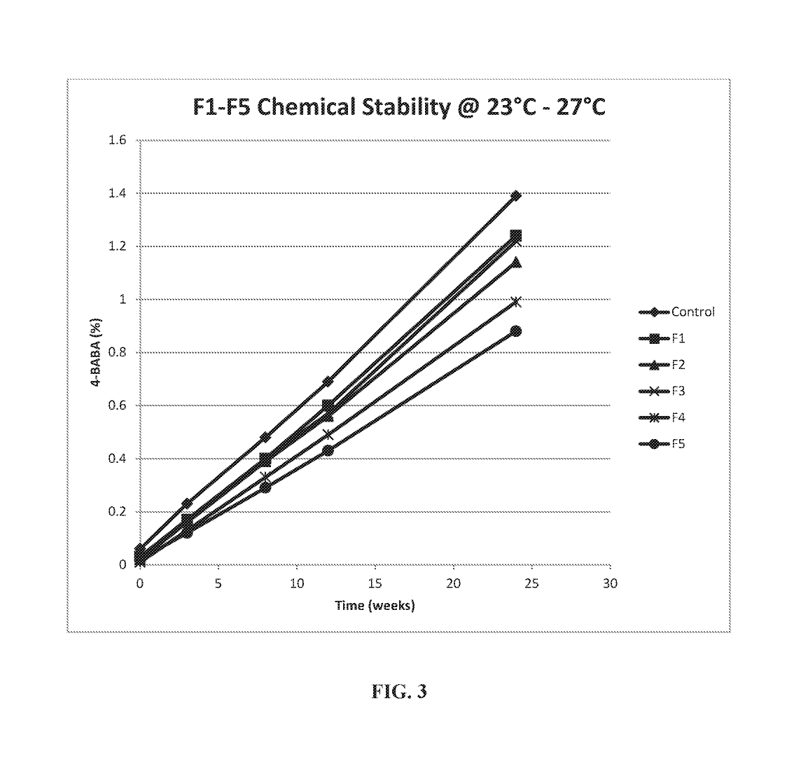

The chemical stability at 25° C.±2° C. for formulations F1-F5 are plotted and shown in FIG. 3.

TABLE 3B
Chemical Stability at 40° C. ± 2° C.

| Storage | C1 | F1 | F2 | F3 | F4 | F5 |
|---|---|---|---|---|---|---|
| (Weeks) | 4-BABA % | | | | | |
| 0 | 0.06 | 0.03 | 0.02 | 0.02 | 0.01 | 0.02 |
| 3 | 0.69 | 0.59 | 0.60 | 0.57 | 0.48 | 0.43 |
| 8 | 1.78 | 1.57 | 1.64 | 1.59 | 1.34 | 1.16 |
| 12 | 2.69 | 2.37 | 2.44 | 2.36 | 2.08 | 1.84 |
| 24 | 5.39 | 4.90 | 5.02 | 5.01 | 4.26 | 3.67 |
| Water | 0 | 2 | 2 | 2 | 6 | 8 |
| Reduction | | | | | | |
| % Change | 0 | −9% | −7% | −7% | −21% | −32% |
| in 4-BABA | | | | | | |
| at 24 weeks | | | | | | |

Note:
4-BABA % is calculate as follows: 4-BABA level (w/w) divided by API (active pharmaceutical ingrediant) level (w/w) × 100. The w/w units cancel out and the unit of measurement is a percentage.

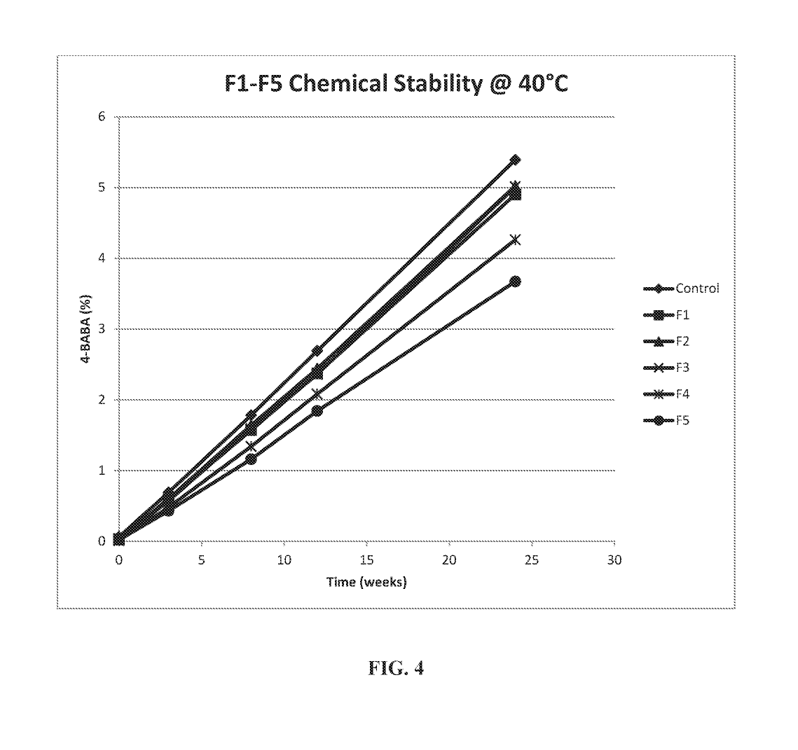

The chemical stability at 40° C.±2° C. for formulations F1-F5 are plotted and shown in FIG. 4.

### Example 4

### Viscosity of the Formulations of Example 1

Each of the formulations and the control described in Example 1 were tested for changes in viscosity following storage at long term conditions (25° C.±2° C./60%±5% RH) and accelerated conditions (40° C.±2° C. and 75%±5% RH). Samples in both long term and accelerated conditions were tested at zero and at various week increments. Viscosity measurements of the formulations were performed with the Brookfield HA DV-11+Pro viscometer using appropriate spindle and rotation speed at a temperature maintained between 23° C.±2° C. The samples were placed in the sample adapter and maintained at the 23° C.±2° C. for 30 minutes before measurement. Observations were recorded every two minutes until two consecutive readings were within ±10 cps. Generally spindle #21 was used for the measurements for formulations with viscosity less than 6,000 cps, spindle #14 was used for viscosities higher than 6,000 cps, and spindle #7 was used for thicker formulations.

Results for the viscosity changes for each of the formulations are provided in Table 4A for the long term temperature samples and Table 4B for the accelerated temperature samples.

TABLE 4A
Viscosity at 25° C. (cps)

| Storage | C1 | F1 | F2 | F3 | F4 | F5 |
|---|---|---|---|---|---|---|
| (Weeks) | Viscosity (cps) | | | | | |
| 0 | 774 | 2,305 | 375,500 | 2,578 | 6,655 | 5,313 |
| 3 | 696 | 1,615 | 236,300 | 2,470 | 8,500 | 18,620 |
| 8 | 642 | 1,246 | 314,300 | 2,420 | 2,825 | 11,800 |
| 12 | 716 | 1,180 | 234,000 | 2,350 | 7,175 | 13,200 |
| 24 | 628 | 1,118 | 142,300 | 2,350 | 9,400 | 13,150 |

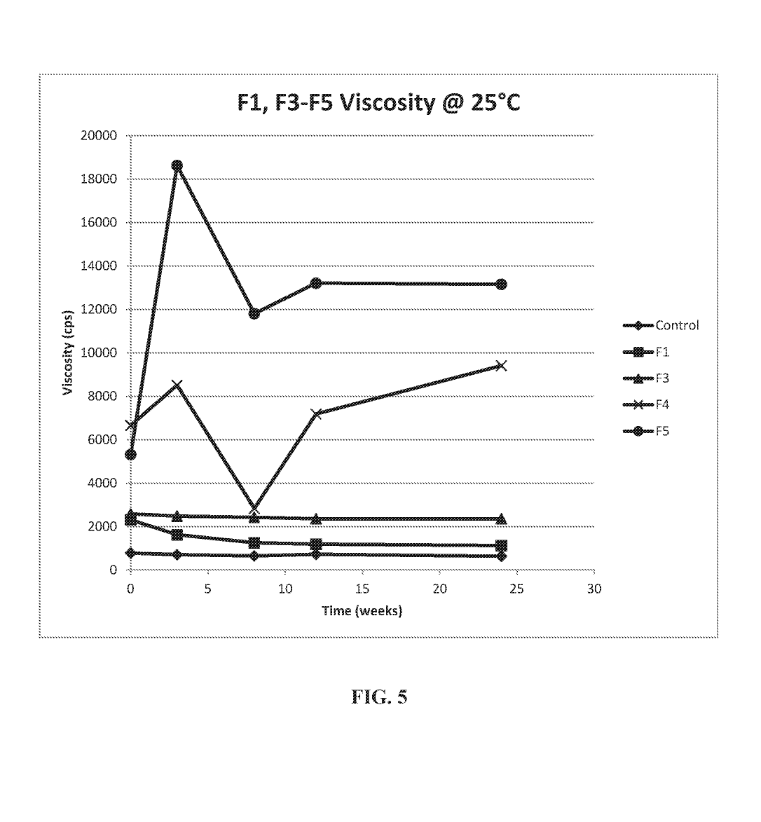

The viscosity at 25° C.±2° C. for formulations F1, F3-F5 are plotted and shown in FIG. 5. Note that formulation F2 is not plotted because its values are so much larger than the other formulations and adding F2 to the graph distorts the plot.

TABLE 4B
Viscosity at 40° C. (cps)

| Storage | C1 | F1 | F2 | F3 | F4 | F5 |
|---|---|---|---|---|---|---|
| (Weeks) | Viscosity (cps) | | | | | |
| 0 | 774 | 2,305 | 375,500 | 2,578 | 6,655 | 5,313 |
| 3 | 762 | 1,536 | 329,300 | 2,520 | 7,900 | 9,840 |
| 8 | 716 | 1,458 | 270,300 | 2,325 | 5,225 | 8,000 |
| 12 | 820 | 1,392 | 240,000 | 2,350 | 7,525 | 9,150 |
| 24 | 852 | 2,020 | 166,300 | 2,400 | 7,650 | 8,350 |

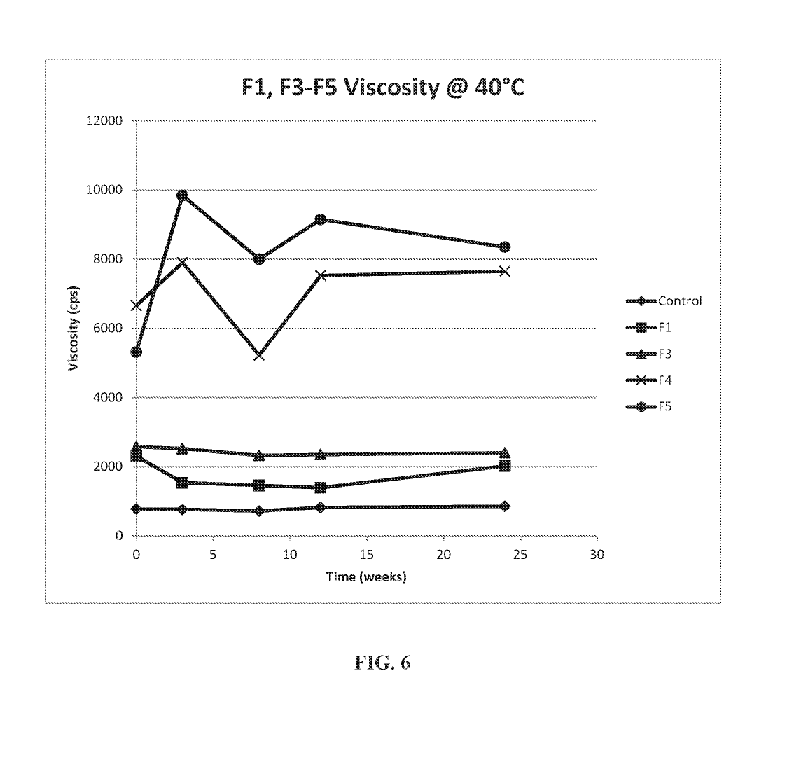

The viscosity at 40° C.±2° C. for formulations F1, F3-F5 are plotted and shown in FIG. 6. As noted above, formulation F2 is not plotted because its values are so much larger than the other formulations and adding F2 to the graph distorts the plot.

### Example 5

### Transdermal System for Delivering Local Anesthetics

Table 5 provides example ingredient ranges for formulations containing lidocaine and tetracaine as local anesthetics and sodium lauryl sulfoacetate. Generally, formulations having the compositional make up set forth in the table can be manufactured in a manner similar to the manufacturing process described in Example 1. The final products provide improved physical and/or chemical stability as compared to those similar products where the sodium lauryl sulfoacetate is replaced with an equivalent amount of water.

TABLE 5
Example Formulation Ranges

| Ingredient | Wt % Ranges |
|---|---|
| Lidocaine | 3 to 25 |
| Tetracaine | 3 to 25 |
| Polyvinyl Alcohol | 2 to 15 |
| Sorbitan Monopalmitate | 1 to 5 |
| Purified Water | 25 to 80 |
| Paraben | 0.01 to 0.6 |
| Sodium Lauryl Sulfoacetate | 0.1 to 30 |

### Example 6

### Transdermal Delivery Formulations—Set 2

Several formulations were prepared according to embodiments of the present disclosure utilizing the compositional components set forth in Table 6. Each of the formulations was prepared in a batch at a batch size of 2 kg. All raw materials were stored at ambient conditions prior to manufacturing of the formulations. Generally, all formulations were manufactured as described below. Specifically, an oil phase was prepared by heating and mixing the mixture of the active agents (local anesthetic bases admixed to form a eutectic mixture) at about 60° C. (50° C.±5° C. for formulations F6-F12 and 55° C.±5° C. for fomulations F13-F15). The parabens, polymer, and fatty acid ester were added sequentially in water while stirring in combination with homogenization at high shear force under heated conditions (−75° C.±5° C.). The temperature of the mixing vessel was then lowered and the oil phase mixed into water and homogenized at room temperature (25° C.±5° C.) until a product without any lumps or crystals was obtained. The final product was then tested for the physical and chemical stability at the noted storage conditions. A control formulation was also prepared that did not include SLSA. The SLSA content in the control formulation was replaced with water. In addition, two formulations were prepared where the SLSA was replaced with oleic acid. This was done to ensure that the SLSA and not the added water was responsible for the improved stability of the local anesthetic.

TABLE 6
Example Formulations F6-F12

| | C2 | | | | | | | |
|---|---|---|---|---|---|---|---|---|
| | (Control) | F6 | F7 | F8 | F9 | F10 | F11 | F12 |
| Ingredient | Wt % | | | | | | | |
| Lidocaine | 20 | 20 | 20 | 20 | 20 | 20 | 20 | 20 |
| Tetracaine | 20 | 20 | 20 | 20 | 20 | 20 | 20 | 20 |
| Polyvinyl | 7.2 | 7.2 | 7.2 | 7.2 | 7.2 | 7.2 | 7.2 | 7.2 |
| Alcohol | | | | | | | | |
| Sorbitan | 3 | 3 | 3 | 3 | 3 | 3 | 3 | 3 |
| Mono- | | | | | | | | |
| palmitate | | | | | | | | |
| Purified | 49.68 | 49.18 | 48.68 | 46.68 | 39.68 | 34.68 | 48.68 | 46.68 |
| Water | | | | | | | | |
| Methyl- | 0.1 | 0.1 | 0.1 | 0.1 | 0.1 | 0.1 | 0.1 | 0.1 |
| paraben | | | | | | | | |
| Propyl- | 0.02 | 0.02 | 0.02 | 0.02 | 0.02 | 0.02 | 0.02 | 0.02 |
| paraben | | | | | | | | |
| Sodium | — | 0.5 | 1 | 3 | 10 | 15 | — | — |
| Lauryl | | | | | | | | |
| Sulfo- | | | | | | | | |
| acetate | | | | | | | | |
| Oleic Acid | — | — | — | — | — | — | 1 | 3 |

### Example 7

### Physical Stability of the Formulations of Example 6

Each of the formulations and the control described in Example 6 were stored at long term conditions (25° C.±2° C. and 60%±5% RH) and accelerated conditions (40° C.±2° C. and 75%±5% RH). The physical stability (i.e. the phase separation of the formulations) of the samples stored at both long term and accelerated conditions was tested at weeks one, two, three, four, eight, and 12.

The phase separation was measured by placing the formulations in 125 ml graduated glass cylinders at long term conditions and accelerated conditions. Phase separation in the formulations was determined by measuring the amount of phase separation at the top of the cylinder in terms of milliliters. The cylinders containing the formulations were not moved during the course of the study.

Results for the phase separation for each of the formulations are provided in Table 7A for the long term temperature samples and in Table 7B for the accelerated temperature samples.

TABLE 7A
Physical Stability at 25° C. ± 2° C.

| Storage | C2 | F6 | F7 | F8 | F9 | F10** | F11 | F12 |
|---|---|---|---|---|---|---|---|---|
| (Weeks) | Phase separation (ml) | | | | | | | |
| 0 | 0 | 0 | 0 | 0 | 0 | separation | 0 | 0 |
| | | | | | | observed* | | |
| 1 | 10 | 0 | 0 | 0 | 0 | 95 | 0 | 0 |
| 2 | 10 | 0 | 0 | 0 | 0 | 90 | 5 | 5 |
| 3 | 10 | Ring* | 0 | 5 | 0 | 90 | 5 | 5 |
| 4 | 10 | Ring* | 0 | 5 | 0 | 70 | 10 | 10 |
| 8 | 5 | Ring* | 0 | 5 | 35** | 60 | 10 | 10 |
| 12 | 5 | Ring* | 0 | 5 | 100** | 55 | 10 | 10 |

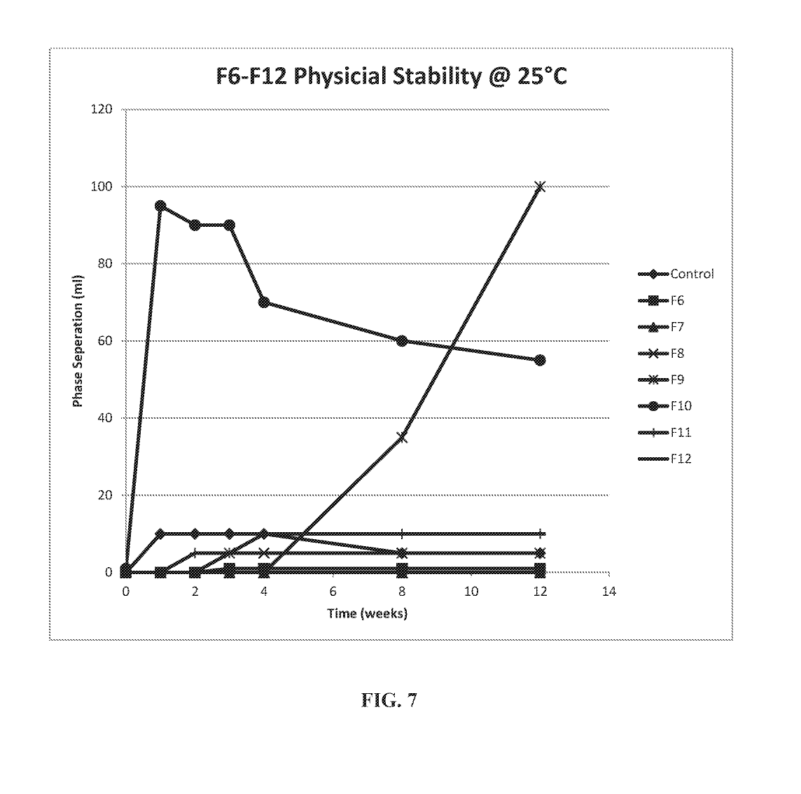

*The ring is a small layer less than 1 ml in size, or the phase seperation observed at t = 0 was not measured. This data is displayed as 1 ml in FIG. 7.
**The values shown represent the total amount of phase separation. These samples included several layers of foam, condensed foam, and/or oil.

The physical stability at 25° C.±2° C. for formulations F6-F12 are plotted and shown in FIG. 7.

TABLE 7B
Physical Stability at 40° C. ± 2° C.

| Storage | C2 | F6 | F7 | F8 | F9 | F10* | F11 | F12 |
|---|---|---|---|---|---|---|---|---|
| (Weeks) | Phase separation (ml) | | | | | | | |
| 0 | 0 | 0 | 0 | 0 | 0 | separation | 0 | 0 |
| | | | | | | observed* | | |
| 1 | 5 | 0.5 | 0 | 0 | 0 | 95 | 10 | 5 |
| 2 | 5 | 0.5 | 0 | 5 | 10 | 100 | 10 | 5 |
| 3 | 10 | 0.5 | 0 | 10 | 10 | 90 | 10 | 5 |
| 4 | 10 | 0.5 | 0 | 10 | 10 | 85 | 5 | 5 |
| 8 | 10 | 0.5 | 0 | 10 | 30** | 75 | 5 | 5 |
| 12 | 10 | 5 | 0 | 10 | 95** | 100 | 5 | 5 |

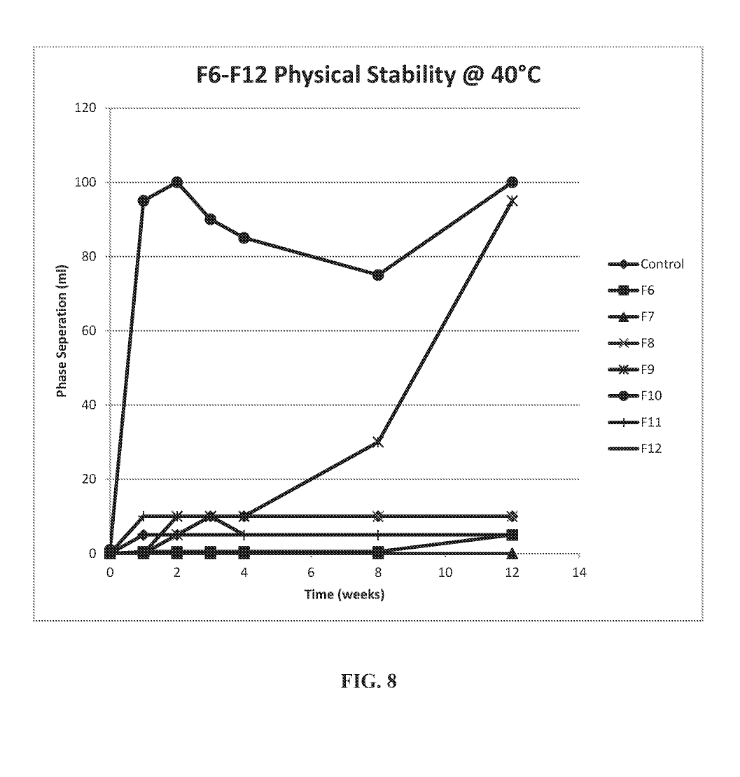

*The phase seperation observed was not measured. This data is displayed as 1 ml in FIG. 8.
**The values shown represent the total amount of phase separation. These samples included several layers of foam, condensed foam, and/or oil.

The physical stability at 40° C.±2° C. for formulations F6-12 are plotted and shown in FIG. 8.

Based on the data in tables 7A and 7B, it was determined that in certain formulations when the amount of SLSA is present at equal to or greater than 15 wt % of the total formulation, that the SLSA can have a negative impact on the physical stability of the composition. Therefore, when an improvement in physical stability is the only formulation improvement sought, depending on the formulation, formulation having less than about 15 wt % SLSA may be more desirable, e.g., from 0.5 wt % to 15 wt % SLSA. However, when other factors are being considered, such as chemical stability or viscosity at specific storage periods, etc., weight percentages outside of this range may likewise be useful in some embodiments. The formulation containing 1 wt % SLSA did not exhibit any phase separation during the example tests and would be a desirable choice if the primary concern is physical stability, though in a broader sense, from 0.5 wt % to 10 wt % would also be desirable for physical stability improvement.

### Example 8

### Chemical Stability of the Formulations of Example 7

Each of the formulations and the control described in Example 7 were tested for the chemical stability of tetracaine following storage at long term conditions (25° C.±2° C. and 60%±5% RH) and accelerated conditions (40° C.±2° C. and 75%±5% RH). The chemical stability was measured in terms of the generation of the impurity 4-butylaminobenzoic acid (4-BABA), the primary hydrolysis degradation product of tetracaine. Specifically, tetracaine breaks down to 4-BABA, so a lower concentration of this compound after a period of weeks demonstrates greater tetracaine chemical stability over time. Samples in both long term and accelerated conditions were tested at zero, four, eight, and 12 weeks.

The chemical stability of the formulations was assessed by measuring the concentration of the degradation products, i.e. impurities, such as 4-BABA using High Performance Liquid Chromatographic (HPLC) method. The HPLC method involved chromatographic separation by mobile phase gradient and C18 analytical column; and quantification of each component by ultraviolet (UV) detector. Results for the chemical stability for each of the formulations are provided in Table 8A for the long term temperature samples and Table 8B for the accelerated temperature samples.

TABLE 8A
Chemical Stability at 25° C. ± 2° C.

| Storage | C2 | F6 | F7 | F8 | F9 | F10 | F11 | F12 |
|---|---|---|---|---|---|---|---|---|
| (Weeks) | 4-BABA % | | | | | | | |
| 0 | 0.11 | 0.10 | 0.09 | 0.08 | 0.07 | 0.04 | 0.06 | 0.05 |
| 4 | 0.34 | 0.32 | 0.29 | 0.25 | 0.21 | 0.13 | 0.26 | 0.25 |
| 8 | 0.56 | 0.54 | 0.50 | 0.41 | 0.34 | 0.23 | 0.47 | 0.45 |
| 12 | 0.79 | 0.76 | 0.70 | 0.58 | 0.47 | 0.38 | 0.68 | 0.65 |

Note:
4-BABA % is calculate as follows: 4-BABA level (w/w) divided by API (active pharmaceutical ingrediant) level (w/w) × 100. The w/w units cancel out and the unit of measurement is a percentage.

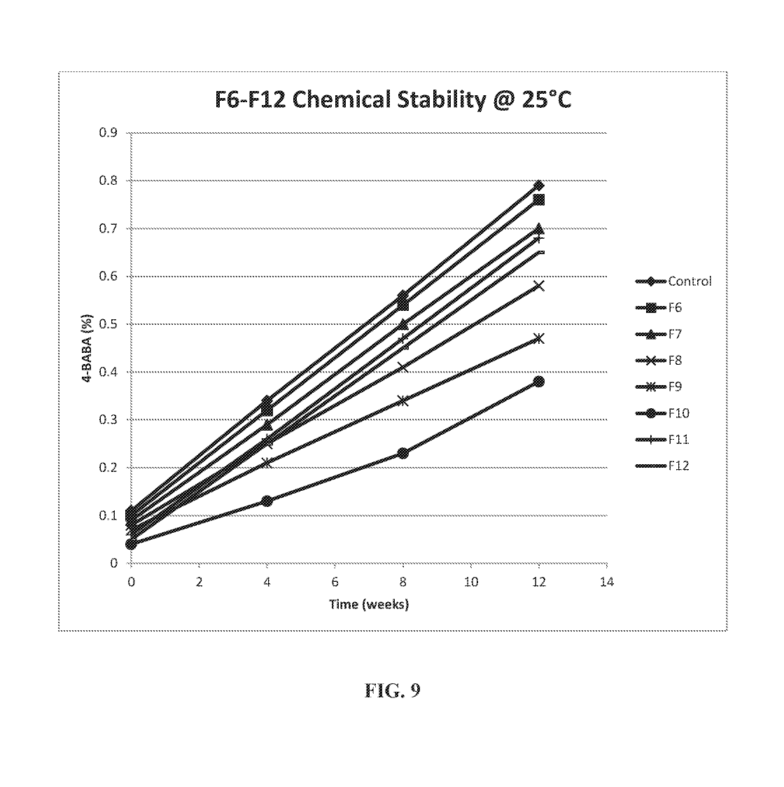

The chemical stability at 25° C.±2° C. for formulations F6-F12 are plotted and shown in FIG. 9.

TABLE 8B
Chemical Stability at 40° C. ± 2° C.

| Storage | C2 | F6 | F7 | F8 | F9 | F10 | F11 | F12 |
|---|---|---|---|---|---|---|---|---|
| (Weeks) | 4-BABA % | | | | | | | |
| 0 | 0.11 | 0.10 | 0.09 | 0.08 | 0.07 | 0.04 | 0.06 | 0.05 |
| 4 | 0.94 | 0.92 | 0.90 | 0.77 | 0.68 | 0.58 | 0.88 | 0.85 |
| 8 | 1.79 | 1.77 | 1.73 | 1.49 | 1.21 | 0.92 | 1.74 | 1.68 |
| 12 | 2.60 | 2.60 | 2.52 | 2.23 | 1.82 | 1.42 | 2.54 | 2.40 |

Note:
4-BABA % is calculate as follows: 4-BABA level (w/w) divided by API (active pharmaceutical ingrediant) level (w/w) × 100. The w/w units cancel out and the unit of measurement is a percentage.

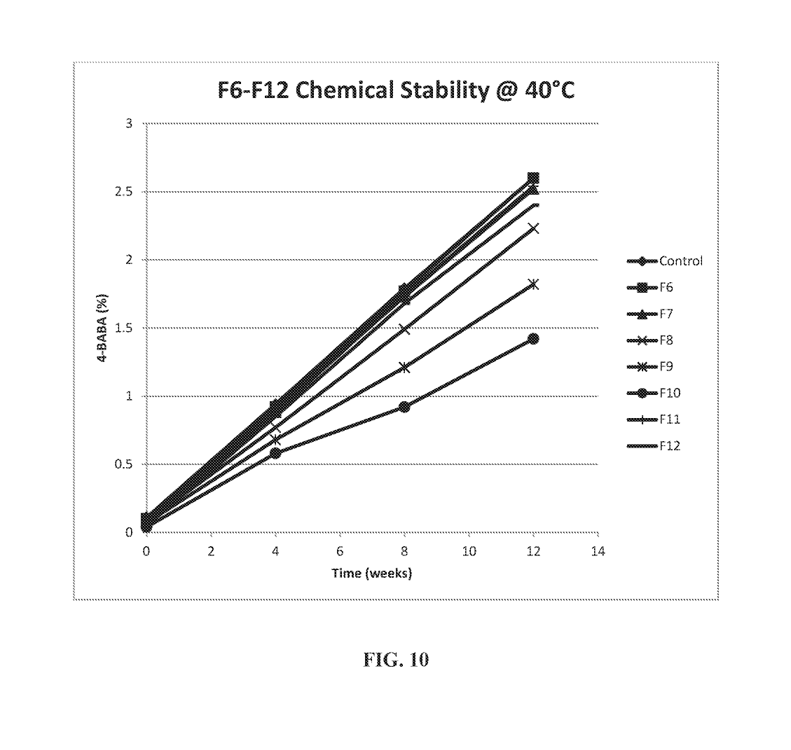

The chemical stability at 40° C.±2° C. for formulations F6-12 are plotted and shown in FIG. 10.

Based on the data in tables 8A and 8B, it was determined that the formulation containing 15 wt % of SLSA exhibited greater chemical stability than formulations containing less than 15 wt % of SLSA. This data confirms that SLSA can be used to improve chemical stability and a formulation containing 15 wt % of SLSA may be a desirable choice when improving physical stability is not as much of an issue. When both chemical and physical stability are a concern, the formulator may be drawn to using a formulation containing less than about 10 wt % SLSA, e.g., from 0.5 wt % to 10 wt %.

### Example 9

### Viscosity of the Formulations of Example 7

Each of the formulations and the control described in Example 7 were tested for changes in viscosity following storage at long term conditions (25° C.±2° C. and 60%±5% RH) and accelerated conditions (40° C.±2° C. and 75%±5% RH). Samples in both long term and accelerated conditions were tested at four, eight, and 12 week increments. Viscosity measurements of the formulations were performed with the Brookfield HA DV-11+Pro viscometer using appropriate spindle and rotation speed. Results for the viscosity changes for each of the formulations are provided in Table 9A for the long term temperature samples and in Table 9B for the accelerated temperature samples.

TABLE 9A
Viscosity (cps) at 25° C. ± 2° C.

| Storage | C2 | F6 | F7 | F8 | F9 | F10 | F11 | F12 |
|---|---|---|---|---|---|---|---|---|
| (Weeks) | Viscosity (cps)* | | | | | | | |
| 4 | 866 | 1,296 | 1,560 | 3,560 | 118,000 | 11,200 | 1,124 | 1,716 |
| 8 | 798 | 1,224 | 1,590 | 3,200 | — | 1,720 | 1,090 | 1,595 |
| 12 | 796 | 1,172 | 1,352 | 3,880 | — | 1,392 | 1,110 | 1,710 |

*The testing conditions (speed/spindle) were varied when obtaining these viscosity measurements; therefore these measurements should be taken as a range and degree of thickness rather than absolute viscosities.

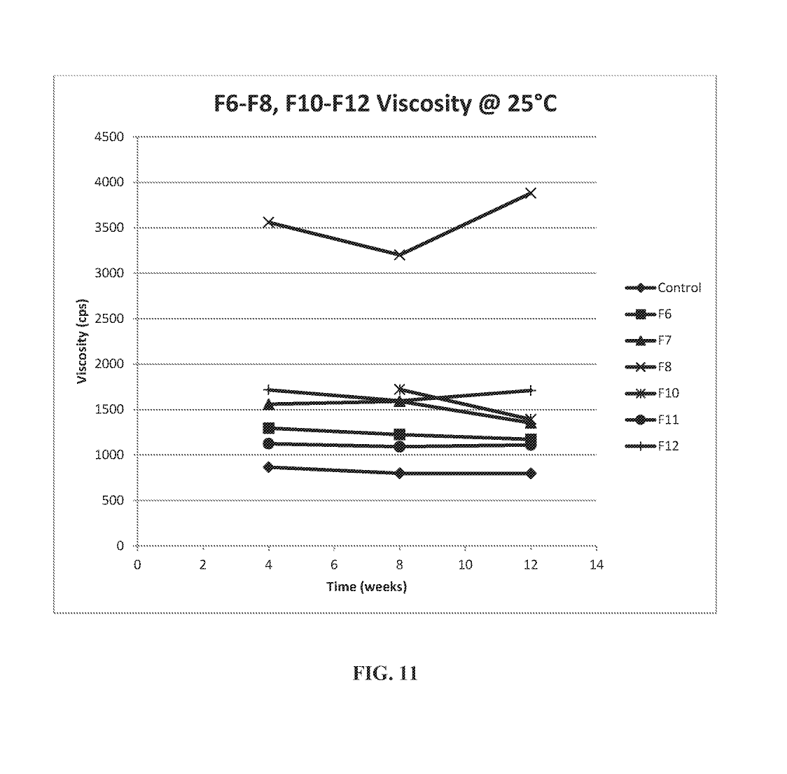

The viscosity at 25° C.±2° C. for formulations F6-F8 and F10-F12 are plotted and shown in FIG. 11. Formulation F9 is not plotted because its values are so much larger than the other formulations and adding F9 to the graph distorts the plot.

TABLE 9B
Viscosity (cps) at 40° C. ± 2° C.

| Storage | C2 | F6 | F7 | F8 | F9 | F10 | F11 | F12 |
|---|---|---|---|---|---|---|---|---|
| (Weeks) | Viscosity (cps)* | | | | | | | |
| 4 | 818 | 1,242 | 1,468 | 4,000 | 148,000 | 58,200 | 1,160 | 1,690 |
| 8 | 796 | 1,124 | 1,384 | 4,200 | 300,000 | — | 1,118 | 1,655 |
| 12 | 822 | 1,172 | 1,344 | 4,700 | — | 440 | 1,094 | 1,840 |

*The testing conditions (speed/spindle) were varied when obtaining these viscosity measurements; therefore these measurements should be taken as a range and degree of thickness rather than absolute viscosities.

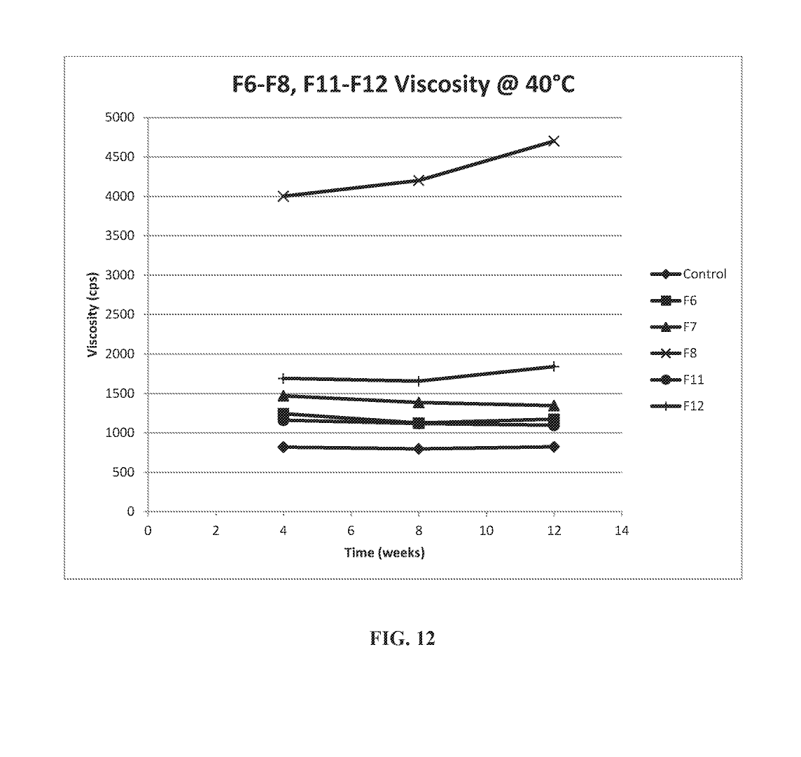

The viscosity at 40° C.±2° C. for formulations F6-F8 and F11-F12 are plotted and shown in FIG. 12. Formulations F9 and F10 are not plotted because their values are so much larger than the other formulations and adding F9 and F10 to the graph distorts the plot.

Based on the data in tables 9A and 9B, it was determined that formulations containing 10 wt % to 15 wt % SLSA exhibited higher viscosities. The viscosities of these formulations were within the range of a spreadable and/or dispensible formulation (28,000 cps to 828,000 cps). Formulations with less than 10 wt % SLSA and with oleic acid in place of the SLSA exhibited a runny consistency and may be more desirable for use in a patch system that provides some structure for the formulation, though the addition of other ingredients, such as polymer may provide desirable consistency for use in a plaster or peel, for example.

### Example 10

### Transdermal Delivery Formulations—Set 3

Several formulations were prepared according to embodiments of the present disclosure utilizing the compositional components set forth in Table 10 below. Each of the formulations was prepared in batches using the methodology described in Example 6. The final product was then tested for the physical-chemical properties and placed on stability at specified storage conditions.

A control formulation was also prepared that did not include SLSA. The SLSA content in the control formulation was replaced with water. In addition, the water content and calcium phosphate content was varied in formulations F14 and F15. This was done because tetracaine is sensitive to hydrolysis so an example without higher concentrations of water was provided to ensure that an increase in water in the control formulation would not significantly impact the results generated by the addition of SLSA.

TABLE 10
Example Formulations F13-F15

| | C3 | | | |
|---|---|---|---|---|
| | (Control) | F13 | F14 | F15 |
| Ingredient | Wt % | | | |
| Lidocaine | 7 | 7 | 7 | 7 |
| Tetracaine | 7 | 7 | 7 | 7 |
| Polyvinyl Alcohol | 14 | 14 | 14 | 14 |
| Sorbitan Monostearate | 4 | 4 | 4 | 4 |
| Calcium Phosphate Dibasic Anhydrous | 27 | 27 | 24 | 26 |
| White Petrolatum | 5 | 5 | 5 | 5 |
| Purified Water | 35.94 | 32.94 | 35.94 | 35.94 |
| Methylparaben | 0.05 | 0.05 | 0.05 | 0.05 |
| Propylparaben | 0.01 | 0.01 | 0.01 | 0.01 |
| Sodium Lauryl Sulfoacetate | — | 3 | 3 | 1 |

### Example 11

### Physical Stability of the Formulations of Example 10

Each of the formulations and the control described in Example 10 were stored at both long term storage conditions (2° C.-8° C.) and accelerated conditions (25° C.±2° C./60%±5% RH). The physical stability (i.e. the phase separation of the formulations) of samples stored at both long term and accelerated storage conditions was tested at one, two, three, four, eight, and 12 weeks.

The phase separation was measured by placing each of the formulations in a 125 ml graduated glass cylinder. The cylinders were stopped and placed at long term and accelerated storage conditions. Phase separation in the formulations were then determined by measuring the quantity of oil phase separated at the top of the cylinder in terms of milliliters. Results for the phase separation for each of the formulations are provided in Tables 11A and 11B.

TABLE 11A
Physical Stability at 2° C. − 8° C.

| | | | | |
|---|---|---|---|---|
| Storage | C3 | F13 | F14 | F15 |

| | |
|---|---|
| (Weeks) | Phase separation (ml) |

| | | | | |
|---|---|---|---|---|
| 0 | 0 | 0 | 0 | 0 |
| 1 | 0 | 0 | 0 | 0 |
| 2 | 0 | 0 | 0 | 0 |
| 3 | 0 | 0 | 0 | 0 |
| 4 | 0 | 0 | 0 | 0 |
| 8 | 0 | 0 | 0 | 0 |
| 12 | 0 | 0 | 0 | 0 |

TABLE 11B
Physical Stability at 25° C. ± 2° C.

| | | | | |
|---|---|---|---|---|
| Storage | C3 | F13 | F14 | F15 |

| | |
|---|---|
| (Weeks) | Phase separation (ml) |

| | | | | |
|---|---|---|---|---|
| 0 | 0 | 0 | 0 | 0 |
| 1 | 0 | 0 | 0 | 0 |
| 2 | 0 | 0 | 0* | 0 |
| 3 | 0 | 0 | 0* | 0 |
| 4 | 0 | 0 | 0* | 0 |
| 8 | 0 | 0 | 0* | 0 |
| 12 | 0 | 0 | 0* | 0 |

*No phase separation was observed; however, there were air bubbles on top of the formulation.

This formulation has high physical stability without the addition of the SLSA. These tests show that the addition of the SLSA does not negatively affect the physical stability of the formulation. This example in combination with Example 12 also shows that SLSA can be added solely to improve the chemical stability of the formulation.

### Example 12

### Chemical Stability of the Formulations of Example 10

Each of the formulations and the control described in Example 10 were tested for the chemical stability of tetracaine at long term storage conditions (2° C.-8° C.) and accelerated conditions (25° C.±2° C. and 60%±5% RH). The chemical stability was measured in terms of the generation of the impurity 4-butylaminobenzoic acid (4-BABA), the primary hydrolysis degradation product of tetracaine. Specifically, tetracaine is known to break down to 4-BABA, so a lower concentration of this compound after a period of weeks demonstrates greater tetracaine stability over time. Samples in both long term storage and accelerated conditions were tested at zero, four, eight, and 12 weeks.

The chemical stability of the formulations was assessed by measuring the concentration of the degradation products, i.e. impurities, such as 4-BABA using High Performance Liquid Chromatographic (HPLC) method. The HPLC method involved chromatographic separation by mobile phase gradient and C18 analytical column and quantification of each component by ultraviolet (UV) detector. Results for the chemical stability for each of the formulations are provided in Table 12A for the long term storage temperature samples and Table 12B for the accelerated temperature samples.

TABLE 12A
Chemical Stability at 2° C.- 8° C.

| | | | | |
|---|---|---|---|---|
| Storage | C3 | F13 | F14 | F15 |

| | |
|---|---|
| (Weeks) | 4-BABA % |

| | | | | |
|---|---|---|---|---|
| 0 | 0.05 | 0.04 | 0.04 | 0.04 |
| 4 | 0.11 | 0.08 | 0.08 | 0.08 |
| 8 | 0.18 | 0.12 | 0.13 | 0.16 |
| 12 | 0.26 | 0.17 | 0.19 | 0.20 |

Note:
4-BABA % is calculate as follows: 4-BABA level (w/w) divided by API (active pharmaceutical ingrediant) level (w/w) × 100. The w/w units cancel out and the unit of measurement is a percentage.

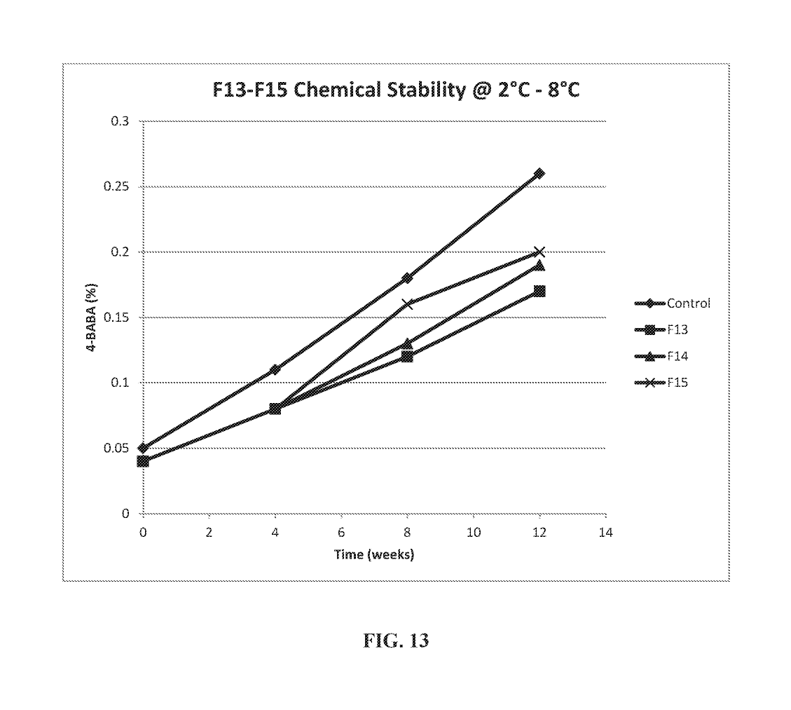

The chemical stability at 5° C. for formulations F13-F15 are plotted and shown in FIG. 13.

TABLE 12B
Chemical Stability at 25° C. ± 2° C.

| | | | | |
|---|---|---|---|---|
| Storage | C3 | F13 | F14 | F15 |

| | |
|---|---|
| (Weeks) | 4-BABA % |

| | | | | |
|---|---|---|---|---|
| 0 | 0.05 | 0.04 | 0.04 | 0.04 |
| 4 | 0.57 | 0.41 | 0.45 | 0.49 |
| 8 | 1.12 | 0.81 | 0.88 | 1 |
| 12 | 1.63 | 1.14 | 1.35 | 1.51 |

Note:
4-BABA % is calculate as follows: 4-BABA level (w/w) divided by API (active pharmaceutical ingrediant) level (w/w) × 100. The w/w units cancel out and the unit of measurement is a percentage.

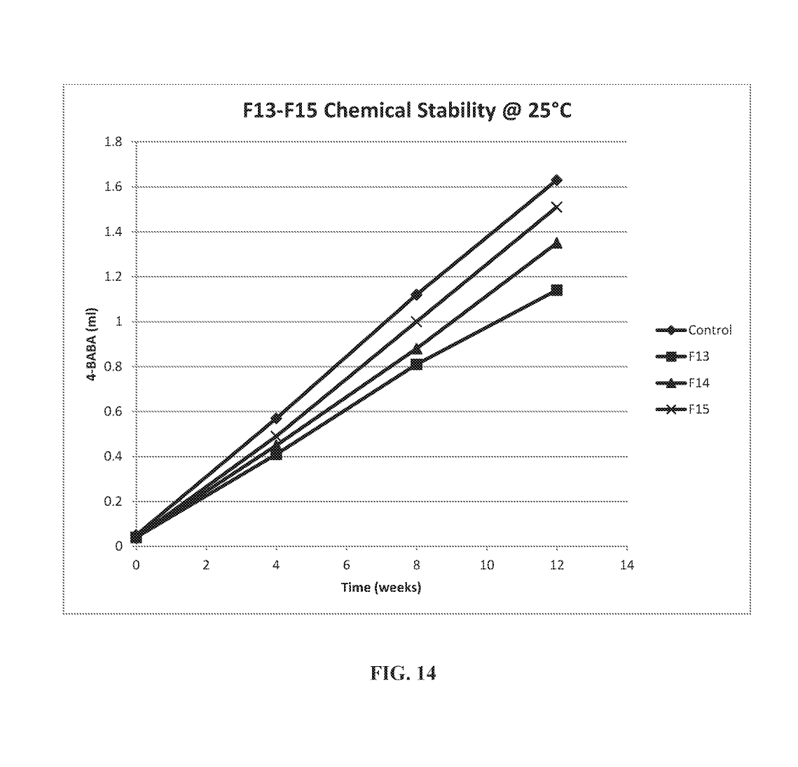

The chemical stability at 25° C.±2° C. for formulations F13-F15 are plotted and shown in FIG. 14.

Decreasing the wt % of the water in the formulation in addition to adding SLSA increased the chemical stability of the formulations. This may be partly because tetracaine is sensitive to hydrolysis. However, it was discovered that a formulation containing 3 wt % SLSA exhibited greater chemical stability than a formulation containing 1 wt % SLSA. Based on this data and the data from Example 8, it appears that within a range, formulations having a greater wt % of SLSA generally have better chemical stability than formulations that contain a lower wt % of SLSA.

### Example 14

### Transdermal System for Delivering Corticosteriods

Table 14 provides example ingredient ranges for formulations containing halobetasol (corticosteriod) and sodium lauryl sulfoacetate. Generally, formulations having the compositional make up set forth in the table can be manufactured in a manner similar to the manufacturing process described in Examples 1 and 6. The final products can provide improved physical and/or chemical stability as compared to those similar products where the sodium lauryl sulfoacetate is replaced with an equivalent amount of water.

TABLE 14
Example Formulation Ranges

| Ingredient | Wt % Ranges |
|---|---|
| Halobetasol | 0.01 to 10 |
| Carrier - Solvent/Emoliant | 10 to 80 |
| Buffer | 0.05 to 2 |
| Water | 10 to 65 |
| Preservative | 0 to 1 |
| Sodium Lauryl Sulfoacetate | 0.1 to 30 |

### Example 15—Transdermal System for Delivering NSAID

Table 15 provides example ingredient ranges for formulations containing Diclofenac (NSAID) and sodium lauryl sulfoacetate. Generally, formulations having the compositional make up set forth in the table can be manufactured in a manner similar to the manufacturing process described in Examples 1 and 6. The final products can provide improved physical and/or chemical stability as compared to those similar products where the sodium lauryl sulfoacetate is replaced with an equivalent amount of water.

TABLE 15
Example Formulation Ranges

| Ingredient | Wt % Ranges |
|---|---|
| Diclofenac | 0.01 to 10 |
| Carrier - Solvent/Emoliant | 10 to 90 |
| Buffer | 0.05 to 2 |
| Water | q.s. |
| Thickner | 0.1 to 5 |
| Sodium Lauryl Sulfoacetate | 0.1 to 30 |

### Example 16

### Transdermal System for Delivering Local Anesthetics

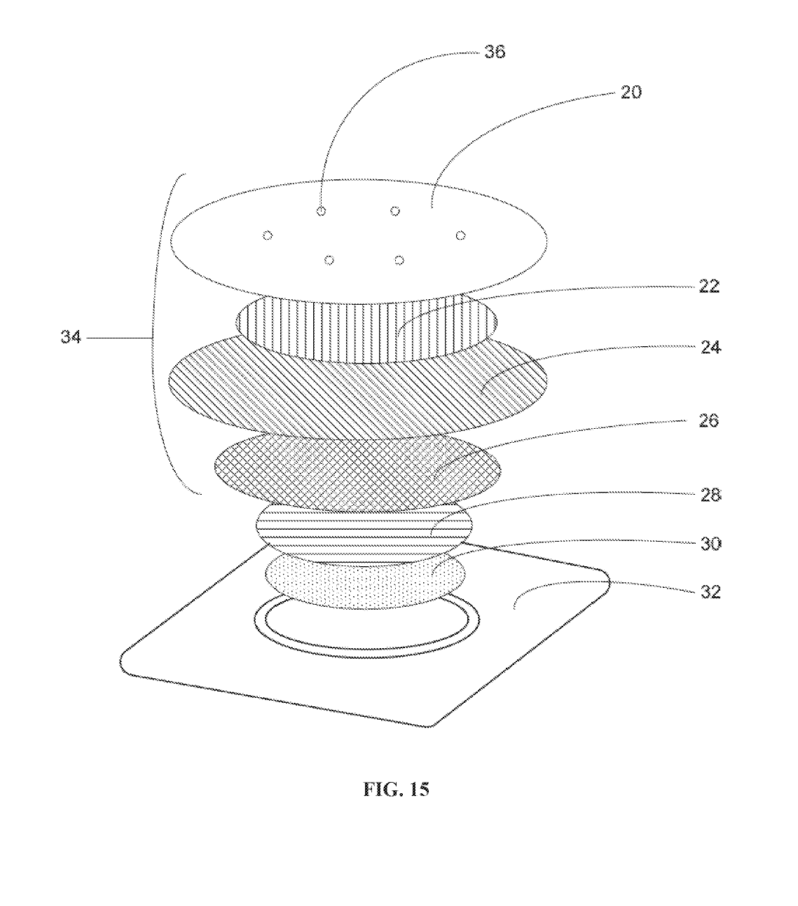

By way of example, a transdermal system that can include a topical formulation where the drug is at least one local anesthetic, NSAID as disclosed herein is shown at FIG. 15. In one specific example, the system can include a heating component 34 and a local anesthetic formulation 30. The heating component can includes an air-impermeable top cover film 20 having a plurality of holes 36 therein. When exposed to ambient air, the holes allow for the passage of the ambient air through the air-impermeable top cover film to the exothermic chemical composition 22. The layer of exothermic chemical composition can be disposed between the air-impermeable top cover film and an adhesive film layer 24. The adhesive film layer extends beyond the circumference of the exothermic chemical composition layer and the local anesthetic formulation layer and can function, at least in part, to adhere to the analgesic system to a skin surface. A heat sealable film layer 26 can be below the adhesive film layer and act to impede the transfer of substances, particularly moisture, between the local anesthetic formulation layer and the exothermic chemical composition layer. Below the heat sealable film layer, a sodium-borate coated non-woven film layer 28 acts to gel the local anesthetic formulation during manufacturing. The topical formulation of the transdermal system can be adhered in an air and moisture impermeable packing tray 32 that holds the local anesthetic formulation during storage. The entire transdermal system can likewise be air sealed in a package to prevent premature activation of the exothermic chemical composition.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

What is claimed is:

1. A topical formulation, comprising:
a drug,
sodium lauryl sulfoacetate, and
water.

2. The topical formulation of claim 1, wherein the drug is an NSAID selected from the group consisting of acetaminophen, aspirin, bromefenac sodium, diclofenac, diclofenac potassium, diclofenac sodium, diflunisal, etodolac, fenoprofen calcium, flurbiprofen, ibuprofen, indomethacin, ketoprofen, magnesium salicylate, meclofenamate sodium, mefenamic acid, nabumetone, naproxen, naproxen sodium, oxaproxin, oxyohebutazone, phenylbutazone, piroxicam, rofecoxib, salsalate, sodium salicylate, sulindac, tometin sodium, valdexocib, and combinations thereof; or a corticosteroid selected from the group of alclometasone dipropionate, amcinonide, beclomethasone dipropionate, betamethasone, betamethasone benzoate, betamethasone dipropionate, betamethasone valerate, budesonide, clobetasol propionate, desonide, desoximetasone, dexamethasone, diflorasone diacetate, fludrocortisone acetate, flunisolide, flurandrenolide, fluocinolone acetonide, fluocinonide, fluticasone propionate, halcinonide, halobetasol, hydrocortisone, hydrocortisone valerate, methylprednisolone, mometasone furoate, prednisolone, prednisone, triamcinolone, triamcinolone acetonide, and combinations thereof; or a local anesthetic selected from the group consisting of lidocaine, tetracaine, benzocaine, prilocaine, bupivacaine, dimethocaine, mepivacaine, procaine, ropivacaine, trimecaine, articaine, and combinations thereof.

3. The topical formulation of claim 1, wherein the drug includes lidocaine, tetracaine, or a combination thereof.

4. The topical formulation of claim 1, wherein the drug includes a eutectic mixture of lidocaine and tetracaine.

5. The topical formulation of claim 1, wherein the drug comprises a local anesthetic and the local anesthetic is at least one local anesthetic base.

6. The topical formulation of claim 1, wherein the drug includes diclofenac.

7. The topical formulation of claim 1, wherein the drug includes halobetasol.

8. The topical formulation of claim 1, wherein the drug comprises at least 14 wt % of the topical formulation.

9. The topical formulation of claim 1, wherein the drug comprises at least about 30 wt % of the topical formulation.

10. The topical formulation of claim 1, wherein the drug comprises at least about 35 wt % of the topical formulation.

11. The topical formulation of claim 1, wherein the sodium lauryl sulfoacetate comprises from about 0.1 wt % to about 30 wt % of the topical formulation.

12. The topical formulation of claim 1, wherein the sodium lauryl sulfoacetate comprises from about 0.5 wt % to about 15 wt % of the topical formulation.

13. The topical formulation of claim 1, wherein the sodium lauryl sulfoacteate comprises less than 15 wt % of the topical formulation.

14. The topical formulation of claim 1, wherein the formulation is a stabilized formulation comprising a stabilizing amount of sodium lauryl sulfoacetate.

15. The topical formulation of claim 14, wherein the topical formulation has less phase separation after two weeks when stored at about 40° C. compared to a comparative formulation devoid sodium lauryl sulfoacetate and replaced with an equivalent wt % of water.

16. The topical formulation of claim 14, wherein the topical formulation has less phase separation after four weeks when stored at about 25° C. compared to a comparative formulation devoid sodium lauryl sulfoacetate and replaced with an equivalent wt % of water.

17. The topical formulation of claim 14, wherein the topical formulation has less phase separation after four weeks when stored at about 40° C. compared to a comparative formulation devoid of sodium lauryl sulfoacetate and replaced with an equivalent wt % of water.

18. The topical formulation of claim 14, wherein the phase separation is at least 10% less after two weeks when stored at about 40° C. compared to a comparative formulation devoid of sodium lauryl sulfoacetate and replaced with an equivalent wt % of water.

19. The topical formulation of claim 14, wherein the phase separation is at least 20% less after two weeks when stored at about 40° C. compared to a comparative formulation devoid of sodium lauryl sulfoacetate and replaced with an equivalent wt % of water.

20. The topical formulation of claim 14, wherein the phase separation is at least 30% less after two weeks when stored at about 40° C. compared to a comparative formulation devoid of sodium lauryl sulfoacetate and replaced with an equivalent wt % of water.

21. The topical formulation of claim 1, further comprising one or more pharmaceutically acceptable excipients.

22. The topical formulation of claim 21, wherein the one or more pharmaceutically acceptable excipients is other than N-lauroyl sarcosine, sodium octyl sulfate, methyl laurate, isopropyl myristate, oleic acid, glyceryl oleate, and combinations thereof.

23. The topical formulation of claim 1, wherein the topical formulation is free of N-lauroyl sarcosine, sodium octyl sulfate, methyl laurate, isopropyl myristate, oleic acid, and glyceryl oleate.

24. The topical formulation of claim 1, further comprising a solidification polymer.

25. The topical formulation of claim 24, wherein the solidification polymer is selected from the group consisting of polyvinyl alcohol, a monobutyl ester of the copolymer of methyl vinyl ether and maleic anhydride, poly(2-hydroxyethyl methacrylate), a copolymer of butyl methacrylate and methyl methacrylate, a copolymer of methacrylic acid and methyl methacrylate, and combinations thereof.

26. The topical formulation of claim 24, wherein the solidification polymer is polyvinyl alcohol.

27. The topical formulation of claim 1, further comprising a sorbitan fatty acid ester.

28. The topical formulation of claim 1, further comprising a paraben.

29. The topical formulation of claim 28, wherein the paraben is selected from the group consisting of methylparaben, propylparaben, ethylparaben, butylparaben, isobutylparaben, isopropyl paraben, benzyl paraben, and combinations thereof.

30. The topical formulation of claim 28, wherein the paraben includes methylparaben, propylparaben, or both.

31. The topical formulation of claim 3, wherein after four weeks stored at about 25° C., the topical formulation has a lower concentration of 4-BABA compared to a comparative formulation devoid of sodium lauryl sulfoacetate and replaced with an equivalent wt % of water.

32. The topical formulation of claim 31, wherein after four weeks stored at about 25° C., the topical formulation has a concentration of 4-BABA that is at least 5% lower when compared to a comparative formulation devoid of sodium lauryl sulfoacetate and replaced with an equivalent wt % of water.

33. The topical formulation of claim 31, wherein after four weeks stored at about 25° C., the topical formulation has a concentration of 4-BABA that is at least 10% lower when compared to a comparative formulation devoid of sodium lauryl sulfoacetate and replaced with an equivalent wt % of water.

34. The topical formulation of claim 1, further comprising a polymer and formulated as a plaster or peel-forming formulation.

35. A method of therapeutically delivering a drug, comprising:
applying a system for transdermal delivery of a drug to a skin surface of a subject, said system comprising a topical formulation, including the drug, sodium lauryl sulfoacetate, and water; and
maintaining the system on the skin surface for a period of time to provide a therapeutic effect to the subject.

36. The method of claim 35, wherein the drug is a local anesthetic and the method is for treating existing pain or preventing pain, and wherein the step of maintaining includes maintaining the system on the skin surface for a period of time of at least 15 minutes such that the topical formulation is in contact with the skin surface to achieve the therapeutic effect.

37. The method of claim 36, wherein the system further comprises a heating component capable of heating the skin surface to a temperature of 32° C. to 47° C. and wherein the method further comprises the step of heating the skin surface and the topical formulation with the heating component.

38. The method of claim 37, wherein the heating component and the topical formulation are integrated into a transdermal patch.

39. The method of claim 37, wherein the heating component begins heating at about the same time as the system is applied to the skin surface.

40. The method of claim 36, wherein the local anesthetic is selected from the group consisting of lidocaine, tetracaine, benzocaine, prilocaine, bupivacaine, dimethocaine, mepivacaine, procaine, ropivacaine, trimecaine, articaine, and combinations thereof.

41. The method of claim 36, wherein the local anesthetic is a local anesthetic base.

42. The method of claim 36, wherein the topical formulation includes two local anesthetics and the two local anesthetics form a eutectic mixture.

43. The method of claim 36, wherein the topical formulation includes lidocaine, tetracaine, or a combination thereof.

44. The method of claim 36, wherein the topical formulation includes a eutectic mixture of lidocaine and tetracaine.

45. The method of claim 44, wherein the weight percentage of the eutectic mixture is at least 30 wt % of the topical formulation.

46. The method of claim 36, wherein the sodium lauryl sulfoacetate comprises from about 0.1 wt % to about 30 wt % of the topical formulation.

47. The method of claim 36, wherein the sodium lauryl sulfoacetate comprises from about 0.5 wt % to about 15 wt % of the topical formulation.

48. The method of claim 36, wherein the system is maintained on the skin surface for a period of time of at least 60 minutes.

49. The method of claim 36, wherein the system is maintained on the skin surface for a period of time of at least two hours.

50. The method of claim 36, wherein the topical formulation has a skin contact region having an area of 2 cm² to 200 cm².

51. The method of claim 36, wherein the topical formulation has a skin contact region having an area of 7 cm² to 150 cm².

52. The method of claim 36, wherein the topical formulation has a skin contact region having an area of 8 cm² to 15 cm².

53. The method of claim 36, wherein the topical formulation has a skin contact region having an area of about 2 cm² to about 12 cm².

54. The method of claim 36, wherein the topical formulation has a skin contact region having an area of about 25 cm² to about 35 cm².

55. The method of claim 36, wherein the topical formulation has a skin contact region having an area of about 15 cm² to about 20 cm².

56. The method of claim 36, wherein the topical formulation has less phase separation after four weeks stored at 25° C. when compared to a comparative formulation devoid of sodium lauryl sulfoacetate and replaced with an equivalent wt % of water.

57. The method of claim 36, wherein the topical formulation further comprises one or more pharmaceutically acceptable excipients.

58. The method of claim 57, wherein the one or more pharmaceutically acceptable excipients is other than N-lauroyl sarcosine, sodium octyl sulfate, methyl laurate, isopropyl myristate, oleic acid, glyceryl oleate, and combinations thereof.

59. The method of claim 36, wherein the topical formulation is free of N-lauroyl sarcosine, sodium octyl sulfate, methyl laurate, isopropyl myristate, oleic acid, and glyceryl oleate.

60. The method of claim 36, wherein the method is for analgesicly treating existing pain.

61. The method of claim 60, wherein the existing pain is musculoskeletal pain, neuropathic pain, headache, connective tissue pain, arthritis pain, or pain associated with injury.

62. The method of claim 60, wherein the existing pain is musculoskeletal pain.

63. The method of claim 60, wherein the existing pain is tendinopathy.

64. The method of claim 36, wherein the method is for anestisizing the skin prior to a painful medical procedure.

65. The method of claim 35, wherein the drug is an NSAID and the method is for analgesically treating existing pain, and wherein the step of maintaining includes maintaining the system on the skin surface for a period of time of at least one hour such that the topical formulation is in contact with the skin surface to achieve the therapeutic effect.

66. The method of claim 65, wherein the period of time is at least 2 hours.

67. The method of claim 65, wherein the period of time is at least 4 hours.

68. The method of claim 65, wherein the NSAID is selected from the group consisting of acetaminophen, aspirin, bromefenac sodium, diclofenac, diclofenac potassium, diclofenac sodium, diflunisal, etodolac, fenoprofen calcium, flurbiprofen, ibuprofen, indomethacin, ketoprofen, magnesium salicylate, meclofenamate sodium, mefenamic acid, nabumetone, naproxen, naproxen sodium, oxaproxin, oxyohebutazone, phenylbutazone, piroxicam, rofecoxib, salsalate, sodium salicylate, sulindac, tometin sodium, valdexocib, and combinations thereof.

69. The method of claim 65, wherein the system further comprises a heating component capable of heating the skin surface to a temperature of 32° C. to 47° C. and wherein the method further comprises the step of heating the skin surface and the topical formulation with the heating component.

70. The method of claim 69, wherein the heating component and the topical formulation are integrated into a transdermal patch.

71. The method of claim 69, wherein the heating component begins heating at about the same time as the system is applied to the skin surface.

72. The method of claim 65, wherein the existing pain is musculoskeletal pain, neuropathic pain, headache, connective tissue pain, arthritis pain, or pain associated with injury.

73. The method of claim 65, wherein the existing pain is musculoskeletal pain.

74. The method of claim 65, wherein the existing pain is tendinopathy.

75. The method of claim 35, wherein the drug is a corticosteroid and the method is for locally treating a skin condition, and wherein the step of maintaining includes maintaining the system on the skin surface for a period of time of at least one hour such that the topical formulation is in contact with the skin surface to achieve the therapeutic effect.

76. The method of claim 75, wherein the corticosteroid is selected from the group of alclometasone dipropionate, amcinonide, beclomethasone dipropionate, betamethasone, betamethasone benzoate, betamethasone dipropionate, betamethasone valerate, budesonide, clobetasol propionate, desonide, desoximetasone, dexamethasone, diflorasone diacetate, fludrocortisone acetate, flunisolide, flurandrenolide, fluocinolone acetonide, fluocinonide, fluticasone propionate, halcinonide, halobetasol, hydrocortisone, hydrocortisone valerate, methylprednisolone, mometasone furoate, prednisolone, prednisone, triamcinolone, triamcinolone acetonide, and combinations thereof.

77. The method of claim 75, further comprising the step of removing the system from the skin site for a duration of time, followed by applying another system back to the skin site for further treatment of the skin condition.

78. The method of claim 75, wherein the system further comprises a heating component capable of heating the skin surface to a temperature of 32° C. to 47° C. and wherein the method further comprises the step of heating the skin surface and the topical formulation with the heating component.

79. The method of claim 78, wherein the heating component and the topical formulation are integrated into a transdermal patch.

80. The method of claim 78, wherein the heating component begins heating at about the same time as the system is applied to the skin surface.

81. The method of claim 75, wherein the skin condition is pruritus, psoriasis, dermatitis, herpetiformis, or eczema.

82. A method of improving the physical stability of topical formulation including a drug, comprising admixing the drug, sodium lauryl sulfoacetate, and water into a common formulation.

83. The method of claim 82, wherein the topical formulation has less phase separation after four weeks stored at about 25° C. when compared to a comparative formulation devoid of sodium lauryl sulfoacetate and replaced with an equivalent wt % of water.

84. The method of claim 82, wherein stability is improved following the storage for a period of at least 2 weeks at a temperature of about 25° C. compared to a comparative formulation devoid of sodium lauryl sulfoacetate and replaced with an equivalent wt % of water.

85. The method of claim 82, wherein the drug is an NSAID selected from the group consisting of acetaminophen, aspirin, bromefenac sodium, diclofenac, diclofenac potassium, diclofenac sodium, diflunisal, etodolac, fenoprofen calcium, flurbiprofen, ibuprofen, indomethacin, ketoprofen, magnesium salicylate, meclofenamate sodium, mefenamic acid, nabumetone, naproxen, naproxen sodium, oxaproxin, oxyohebutazone, phenylbutazone, piroxicam, rofecoxib, salsalate, sodium salicylate, sulindac, tometin sodium, valdexocib, and combinations thereof; or a corticosteroid selected from the group of alclometasone dipropionate, amcinonide, beclomethasone dipropionate, betamethasone, betamethasone benzoate, betamethasone dipropionate, betamethasone valerate, budesonide, clobetasol propionate, desonide, desoximetasone, dexamethasone, diflorasone diacetate, fludrocortisone acetate, flunisolide, flurandrenolide, fluocinolone acetonide, fluocinonide, fluticasone propionate, halcinonide, halobetasol, hydrocortisone, hydrocortisone valerate, methylprednisolone, mometasone furoate, prednisolone, prednisone, triamcinolone, triamcinolone acetonide, and combinations thereof; or a local anesthetic selected from the group consisting of lidocaine, tetracaine, benzocaine, prilocaine, bupivacaine, dimethocaine, mepivacaine, procaine, ropivacaine, trimecaine, articaine, and combinations thereof.

86. The method of claim 82, wherein the drug is a local anesthetic base.

87. The method of claim 82, wherein the drug includes lidocaine, tetracaine, or a combination thereof.

88. The method of claim 82, wherein the drug includes a eutectic mixture of lidocaine base and tetracaine base.

89. The method of claim 82, wherein the drug includes diclofenac.

90. The method of claim 82, wherein the drug includes halobetasol.

91. The method of claim 82, wherein the drug includes local anesthetic comprising at least about 30 wt % of the topical formulation.

92. The method of claim 82, wherein the drug includes local anesthetic comprising at least about 14 wt % of the topical formulation.

93. The method of claim 82, wherein the topical formulation further comprises a polymer.

94. The method of claim 82, wherein the topical formulation further comprises a fatty acid ester.

95. The method of claim 82, wherein the topical formulation further comprises a paraben.

96. The method of claim 82, wherein the topical formulation has less phase separation after four weeks stored at about 40° C. compared to a comparative formulation devoid of the sodium lauryl sulfoacetate and replaced with an equivalent wt % of water.

97. The method of claim 82, wherein the sodium lauryl sulfoacetate comprises from about 0.1 wt % to about 30 wt % of the topical formulation.

98. The method of claim 82, wherein the sodium lauryl sulfoacetate comprises from about 0.5 wt % to about 15 wt % of the topical formulation.

99. The method of claim 82, wherein the topical formulation further comprises one or more pharmaceutically acceptable excipients.

100. The method of claim 99, wherein the one or more pharmaceutically acceptable excipients is other than N-lauroyl sarcosine, sodium octyl sulfate, methyl laurate, isopropyl myristate, oleic acid, glyceryl oleate, and combinations thereof.

101. The method of claim 82, wherein the topical formulation is free of N-lauroyl sarcosine, sodium octyl sulfate, methyl laurate, isopropyl myristate, oleic acid, and glyceryl oleate.

102. A system for transdermal delivery of a drug, comprising:
a topical formulation, including a drug, sodium lauryl sulfoacetate, and water; and
a heating component associated with the topical formulation, said heating component capable of heating the skin surface to a temperature of 32° C. to 47° C.

103. The system of claim 102, wherein the drug is a non-steriodal anti-inflammatory drug (NSAID) selected from the group consisting of acetaminophen, aspirin, bromefenac sodium, diclofenac, diclofenac potassium, diclofenac sodium, diflunisal, etodolac, fenoprofen calcium, flurbiprofen, ibuprofen, indomethacin, ketoprofen, magnesium salicylate, meclofenamate sodium, mefenamic acid, nabumetone, naproxen, naproxen sodium, oxaproxin, oxyohebutazone, phenylbutazone, piroxicam, rofecoxib, salsalate, sodium salicylate, sulindac, tometin sodium, valdexocib, and combinations thereof.

104. The system of claim 102, wherein the drug is a or a corticosteroid selected from the group consisting of alclometasone dipropionate, amcinonide, beclomethasone dipropionate, betamethasone, betamethasone benzoate, betamethasone dipropionate, betamethasone valerate, budesonide, clobetasol propionate, desonide, desoximetasone, dexamethasone, diflorasone diacetate, fludrocortisone acetate, flunisolide, flurandrenolide, fluocinolone acetonide, fluocinonide, fluticasone propionate, halcinonide, halobetasol, hydrocortisone, hydrocortisone valerate, methylprednisolone, mometasone furoate, prednisolone, prednisone, triamcinolone, triamcinolone acetonide, and combinations thereof.

105. The system of claim 102, wherein the drug is a local anesthetic selected from the group consisting of lidocaine, tetracaine, benzocaine, prilocaine, bupivacaine, dimethocaine, mepivacaine, procaine, ropivacaine, trimecaine, articaine, and combinations thereof.

106. The system of claim 102, wherein the drug is at least one local anesthetic and the at least one local anesthetic includes lidocaine, tetracaine, or a combination thereof.

107. The system of claim 102, wherein the drug is at least one local anesthetic and the at least one local anesthetic is a eutectic mixture of lidocaine base and tetracaine base.

108. The system of claim 107, wherein the weight percentage of the eutectic mixture is at least 30 wt % of the topical formulation.

109. The system of claim 102, wherein the drug is at least one local anesthetic base.

110. The system of claim 102, wherein the drug is at least one local anesthetic and the local anesthetic comprises at least about 14 wt % of the topical formulation.

111. The system of claim 102, wherein the drug comprises at least about 30 wt % of the topical formulation.

112. The system of claim 102, wherein the drug comprises at least about 35 wt % of the topical formulation.

113. The system of claim 102, wherein the drug is at least one NSAID.

114. The system of claim 113, wherein the NSAID is diclofenac.

115. The system of claim 102, wherein the drug is a corticosteroid.

116. The system of claim 102, wherein the formulation is a stabilized formulation comprising a stabilizing amount of sodium lauryl sulfoacetate.

117. The system of claim 102, wherein the topical formulation has less phase separation after four weeks stored at about 25° C. when compared to a comparative formulation devoid of sodium lauryl sulfoacetate and replaced with an equivalent wt % of water.

118. The system of claim 102, wherein the topical formulation has less phase separation after two weeks when stored at about 40° C. when compared to a comparative formulation devoid of sodium lauryl sulfoacetate and replaced with an equivalent wt % of water.

119. The system of claim 102, wherein the phase separation is at least 10% less after two weeks when stored at about 40° C. when compared to a comparative formulation devoid of sodium lauryl sulfoacetate and replaced with an equivalent wt % of water.

120. The system of claim 102, wherein the sodium lauryl sulfoacetate comprises from about 0.1 wt % to about 30 wt % of the topical formulation.

121. The system of claim 102, wherein the sodium lauryl sulfoacetate comprises from about 0.5 wt % to about 15 wt % of the topical formulation.

122. The system of claim 102, wherein the topical formulation further comprises one or more pharmaceutically acceptable excipients.

123. The system of claim 102, wherein the one or more pharmaceutically acceptable excipients is other than N-lauroyl sarcosine, sodium octyl sulfate, methyl laurate, isopropyl myristate, oleic acid, glyceryl oleate, and combinations thereof.

124. The system of claim 102, wherein the topical formulation is free of N-lauroyl sarcosine, sodium octyl sulfate, methyl laurate, isopropyl myristate, oleic acid, and glyceryl oleate.

125. The system of claim 102, wherein the topical formulation further comprises a solidification polymer selected from the group consisting of polyvinyl alcohol, a monobutyl ester of the copolymer of methyl vinyl ether and maleic anhydride, poly(2-hydroxyethyl methacrylate), a copolymer of butyl methacrylate and methyl methacrylate, a copolymer of methacrylic acid and methyl methacrylate, and combinations thereof.

126. The system of claim 102, wherein the topical formulation further comprises a sorbitan fatty acid ester.

127. The system of claim 102, wherein the topical formulation further comprises a paraben selected from the group consisting of methylparaben, propylparaben, ethylparaben, butylparaben, isobutylparaben, isopropyl paraben, benzyl paraben, and combinations thereof.

128. The system of claim 102, wherein the drug comprises tetracaine, and after four weeks stored at about 25° C. the topical formulation has a lower concentration of 4-BABA when compared to a comparative formulation devoid sodium lauryl sulfoacetate and replaced with an equivalent wt % of water.

129. The system of claim 102, wherein the drug comprises tetracaine, after four weeks stored at about 25° C., the topical formulation has a concentration of 4-BABA that is at least 5% lower when compared to a comparative formulation devoid of sodium lauryl sulfoacetate and replaced with an equivalent wt % of water.

130. The system of claim 102, wherein the heating component includes an exothermic heating material.

131. The system of claim 102, wherein the heating component includes an exothermic chemical composition layer.

132. The system of claim 102, wherein the heating component includes an air impermeable layer disposed on an upper surface of the chemical composition layer and having one or more holes therein.

133. The system of claim 132, wherein the heating component includes an activation tab removably adhered to an upper surface of the air impermeable layer and being configured to cover the one or more holes in the air impermeable layer and inhibit the passage of air through the holes prior to removal of the activation tab.

134. The system of claim 132, wherein the system includes an adhesive layer disposed on a lower surface of one or both of the exothermic chemical composition layer and the lower surface of the air impermeable layer, said adhesive layer being configured to at least assist in adhering the system to a skin surface.

135. The system of claim 102, wherein the heating component is capable of heating the skin surface to a temperature of generates a controlled level of heat from about 36° C. to about 42° C.

136. The system of claim 102, wherein the topical formulation has a skin contact region having an area of 2 cm² to 200 cm².

137. The system of claim 102, wherein the topical formulation has a skin contact region having an area of 7 cm² to 150 cm².

138. The system of claim 102, wherein the topical formulation has a skin contact region having an area of 8 cm² to 15 cm².

139. The system of claim 102, wherein the topical formulation has a skin contact region having an area of about 2 cm² to about 12 cm².

140. The system of claim 102, wherein the topical formulation has a skin contact region having an area of about 25 cm² to about 35 cm².

141. The system of claim 102, wherein the topical formulation has a skin contact region having an area of about 15 cm² to about 20 cm².

142. The system of claim 102, wherein the system is substantially oval, round, square, triangular, or rectangular in shape.

143. A process for stabilizing a topical formulation comprising admixing a stabilizing amount of sodium lauryl sulfoacetate with a drug and water to form a stabilized topical formulation.

144. A stabilized topical formulation derived from the process of claim 143.
